(19) United States
(12) Patent Application Publication
(10) Pub. No.: US 2026/0090923 A1
(43) Pub. Date: Apr. 2, 2026

(54) COLORED CELLULOSIC TAMPON APPLICATOR

(71) Applicant: THE PROCTER & GAMBLE COMPANY, Cincinnati, OH (US)

(72) Inventors: Khalid QURESHI, Mason, OH (US); Ryo MINOGUCHI, Cincinnati, OH (US); Masaharu NISHIKAWA, Cincinnati, OH (US); Paul Thomas WEISMAN, Cincinnati, OH (US)

(21) Appl. No.: 19/345,653

(22) Filed: Sep. 30, 2025

Related U.S. Application Data
(60) Provisional application No. 63/701,858, filed on Oct. 1, 2024.

(51) Int. Cl. A61F 13/26

(52) U.S. Cl. CPC A61F 13/26

(57) ABSTRACT

A tampon product is provided including an applicator comprising an outer member and an inner member slidably engaged with the outer member. The outer member comprises an outer surface and a barrel region. The outer member comprises two or more layers, where an outer layer of the two or more layers forms at least a portion of the outer surface of the outer member. The outer layer comprises cellulosic material. At least a portion of the outer surface of the outer member has a first color, where the first color has an L* value of less than 65 and a gloss level of greater than 5.

### CROSS REFERENCE TO RELATED APPLICATIONS

This application claims the benefit, under 35 U.S.C. § 119 (c), to U.S. Provisional Application No. 63/701,858, filed Oct. 1, 2024, the entire disclosure of which is fully incorporated by reference herein.

### FIELD

The present disclosure relates to tampon applicators, and more particularly, to tampon applicators made of cellulosic material and having one or more colors.

### BACKGROUND

Consumer products or portions thereof that have components derived from petroleum (such as but not limited to plastics), are currently becoming subject to increased regulation by governments in a number of countries, and growing disfavor in some segments of the consumer market. Some reasons for this relate generally to the consumer perception of certain materials having a relatively adverse impact on the environment.

Tampons have been used for many years to manage menstrual discharge. Many consumers prefer tampons provided inside disposable applicators (rather than tampons configured to be inserted via a finger (“digitally”) without use of an applicator). The typical tampon applicator is configured to house and protect the new tampon from deformation and/or contamination prior to use, and then to facilitate application of the tampon when its use is desired.

Currently, many tampon applicators or parts thereof are formed of thermoplastics such as polyethylene. The properties of thermoplastics and known associated forming/molding technologies enable a manufacturer to efficiently make an applicator having relatively sophisticated features, which may be provided for purposes of functionality, convenience, comfort during use, ergonomics and/or aesthetic appeal, thereby making it attractive and convenient to consumers. FIG. 1A depicts a conventional plastic applicator 10 with a barrel region 12 having an external diameter 13, a plunger 30 having an external diameter 31 and a grip region 18 positioned between the barrel region 12 and plunger 30. As shown in FIG. 1A, the external diameter 31 of the plunger 30 is noticeably less than the external diameter 13 of the barrel region 12. These conventional plastic applicators are available in a variety of colors.

Other types of tampon applicators currently marketed are formed of paper or cardboard. While such applicators reduce reliance on and use of materials derived from petroleum as compared to plastic applicators, currently, these cardboard applicators do not provide the same functionality, comfort during use, ergonomics and/or aesthetic appeal as plastic applicators. FIG. 1B depicts a conventional cardboard applicator 10′ including a barrel region 12′ having an external diameter 13′, a plunger 30′ having an external diameter 31′ and a grip region 18′ between the barrel region 12′ and the plunger 30′. Unlike the plastic applicator 10 of FIG. 1A, the external diameter 31′ of the plunger 30′ is relatively close the external diameter 13′ of the barrel region 12′. FIG. 1C depicts another conventional cardboard applicator 10′ where the grip region 18′ includes four impressions which are spaced apart by the outer surface 42′ of the grip region 18′. Unlike the conventional plastic applicator, the conventional cardboard applicator are only available in a limited number of colors (e.g., white, light pink).

Accordingly, there remain opportunities for improvements in cardboard applicators design and functionality such that carboard applicators more closely emulate their plastic applicator counterparts.

### SUMMARY

The inventors of the present invention recognized various advantages of the conventional plastic applicator that are absent in conventional paper or cardboard applicators. These include the features of the plastic applicators which facilitate functionality, convenience, comfort during use, ergonomics and aesthetic appeal. The inventors also recognized that recent environmental regulations in various geographical territories that promote the use of cardboard material over plastic material will likely cause a shift in consumer demand towards cardboard applicators. Thus, the inventors developed an improved cardboard applicator herein, which achieves many of the advantages of plastic applicators that are absent in conventional cardboard applicators. In order to achieve this improved cardboard applicator, the inventors necessarily overcame various design obstacles not encountered in manufacturing plastic applicators.

The inventors of the present invention also recognized other advantages of the conventional plastic applicator that are absent in conventional cardboard applicators. These include that the conventional plastic applicators are available in a variety of colors (e.g., dark blue, magenta, light green, bring pink, etc.) in which the conventional cardboard applicators are not available. The inventors recognized various reasons limiting the color availability of conventional cardboard applicators, including that the nature of the cardboard material itself (e.g., including multiple cellulosic layers) and how the cardboard material is formed (e.g., wound together to form a scam such as a spiral or non-linear shaped seam). Thus, the inventors developed an improved cardboard applicator herein, which is available in a variety of colors not available with conventional cardboard applicators. In order to achieve this improved cardboard applicator available in a variety of colors, the inventors necessarily overcame various design obstacles in manufacturing cardboard applicators with a variety of colors that are not encountered when manufacturing plastic applicators.

In some embodiments, a tampon product is provided including an applicator comprising an outer member and an inner member slidably engaged with the outer member. The outer member comprises an outer surface and a barrel region. The outer member comprises two or more layers, where an outer layer of the two or more layers forms at least a portion of the outer surface of the outer member. The outer layer comprises cellulosic material. At least a portion of the outer surface of the outer member has a first color, where the first color has an L* value of less than 65 and a gloss level of greater than 5.

In some embodiments, a tampon product is provided including an applicator comprising an outer member and an inner member slidably engaged with the outer member. The outer member comprises an outer surface and a barrel region. The outer member comprises two or more layers, where an outer layer of the two or more layers forms at least a portion of the outer surface of the outer member. The outer layer comprises cellulosic material. At least a portion of the outer surface of the outer member has a gloss level of greater than 5. At least a portion of the outer layer has a first color, where the first color has an a* value and a b* value. The b* value satisfies any one of:

$${b}^{*} value > 3.75 \times {a}^{*} value + 9.75$$

$${b}^{*} value > 0.19 \times {a}^{*} value + 6.19$$

$${b}^{*} value < 0.71 \times {a}^{*} value - 5.4 5 .$$

In some embodiments, a tampon product is provided that includes an applicator comprising an outer member and an inner member slidably engaged with the outer member. The outer member comprises an outer surface and a barrel region. The outer member comprises two or more layers, where an outer layer forms at least a portion of the outer surface of the outer member. The outer layer comprises cellulosic material. At least a portion of the outer surface of the outer member has a gloss level greater than 5. At least a portion of the outer layer has a first color, where the first color has an a* value and a b* value. The a* value and the b* value of the first color are outside a boundary defined by equations:

$${b}^{*} value = 3.75 \times {a}^{*} value + 9.75$$

$${b}^{*} value = 0.19 \times {a}^{*} value + 6.19$$

$${b}^{*} value = 0.71 \times {a}^{*} value - 5.4 5 .$$

### BRIEF DESCRIPTION OF THE DRAWINGS

FIG. 1A is a front view of a conventional plastic applicator including a barrel region and a plunger.

FIG. 1B is a front view of a conventional cardboard applicator including a barrel region and a plunger.

FIG. 1C is a front view of a conventional cardboard applicator.

FIG. 2A is a perspective view of a tampon product with an applicator including a barrel region and a plunger.

FIG. 2B is a longitudinal side view of the tampon product of FIG. 2A taken along the line 2B-2B.

FIG. 2C is a longitudinal side view of a plunger of the tampon product of FIG. 2A with a flange at a second end region thereof.

FIG. 2D is a perspective view of a plunger of the tampon product with a flange at a first end region and second end region thereof.

FIG. 3A is a perspective view of a machine used to form circumferential faces in the grip region.

FIG. 3B is a perspective view of a plurality of forming members of the machine of FIG. 3A that move radially inward to form the circumferential faces in the grip region of the applicator.

FIGS. 4A through 4C are perspective and top views of a plurality of circumferential faces and ribs therebetween formed in the grip region.

FIG. 4D is a cross-sectional view of the grip region of FIG. 4A taken along the line 4D-4D.

FIG. 4E is a cross-sectional view of a rib of the cross-sectional view of the grip region of FIG. 4D.

FIG. 4F is a cross-sectional view of a rib of the cross-sectional view of the grip region.

FIG. 4G is a cross-sectional view of a grip region.

FIG. 4H is a cross-sectional view of a grip region.

FIGS. 5A through 5C are perspective views of an end region of the barrel region.

FIGS. 6A through 6C are side views of an applicator including a barrel region and a plunger with circumferential faces formed in both the barrel region and the plunger.

FIG. 7A is a cross-sectional side view of a plurality of material layers that define the applicator.

FIG. 7B is a side view of an applicator including a spirally wound barrel region.

FIG. 7C is a side view of an applicator including a convolutely wound barrel region.

FIG. 7D is a cross-sectional side view of a plurality of material layers that define the applicator with a coating on an outer surface of the first layer and an adhesive layer between first and second layers.

FIG. 8A is a top perspective view of a 3D dimension space of the Lab Color Space.

FIG. 8B is a top view of a 2D dimensional a*-b* plane of the 3D dimensional space of FIG. 8A.

FIG. 8C is a side perspective view of the 3D dimensional space of FIG. 8A with axes indicating the a* values, b* values and L* values of the L*-a*-b* Color Space.

FIG. 9 is a graph that indicates various a*-b* values of different plastic and carboard materials used to make conventional applicators.

FIG. 10A is a side view of a tampon product with a colored applicator including a barrel region and a plunger.

FIG. 10B is a perspective top view of the forward end of the barrel region of the colored applicator of FIG. 10A.

FIG. 10C is a perspective bottom view of the plunger of the colored applicator of FIG. 10A.

FIG. 11 is a side view of a tampon product with a multi-colored applicator including a barrel region and a plunger.

FIG. 12A is a side view of a tampon product with a colored applicator and indicia on an outer surface thereof and oriented parallel to a seam defining the outer surface.

FIG. 12B is a side view of a tampon product with a colored applicator and indicia on an outer surface thereof and extending across a seam defining the outer surface.

FIG. 12C is a side view of the colored applicator of FIG. 12A where the indicia forms an angle with a longitudinal applicator axis.

### DETAILED DESCRIPTION

The following term explanations may be useful in understanding the present disclosure.

As used herein the term “tampon” refers to any type of absorbent structure which is configured to be inserted into the vaginal cavity for the interception and absorption of fluid therefrom. Typically, a tampon includes a pledget structure including a quantity of absorbent material, often absorbent fibrous material, which pledget structure has been bunched, folded and/or compressed in one or more lateral/radial directions, the longitudinal direction, or both, via application of pressure, heat and/or moisture control, in order to provide a formed tampon having a size, shape (typically approximately cylindrical) and stability of form to facilitate insertion into the vagina. A tampon which has been so formed is referred to herein has a “self-sustaining” form. The degree of compression, heat and moisture control applied to the pledget is sufficient such that in the subsequent absence of the external forces and absence of substantial contact with moisture, the pledget will tend to retain its general formed shape and size. It will be understood by persons of ordinary skill in the art that this self-sustaining form typically does not persist following insertion of the tampon. Once the tampon is inserted and begins to contact and absorb fluid, the pledget will swell with absorbed fluid and lose its self-sustaining form.

As used herein the terms “pledget” or “tampon pledget” are intended to be interchangeable and refer to a structure including absorbent material configured to perform the primary function of the tampon, absorption of menstrual fluid. A tampon pledget is sometimes referred to as a tampon blank, or a softwind, and the term “pledget” is intended to include structures designated by such terms as well.

As used herein “vaginal cavity” refers to the internal space within the genitalia of the human female, located between the introitus of the vagina (sometimes referred to as the sphincter of the vagina) and the cervix.

With respect to a tampon and an applicator, the “longitudinal” direction is the ordinary general direction of ejection from an applicator; and also corresponds with the ordinary general direction of insertion of a tampon and applicator into, and their withdrawal from, the vaginal cavity in normal use. For a completely manufactured, pre-use tampon that has a pledget with a generally cylindrical or capsule-shaped self-sustaining form, the longitudinal axis of the form lies generally or approximately along the longitudinal direction. A “radial” or “circumferential” direction is a direction perpendicular to the longitudinal direction. The “circumferential” direction is perpendicular to the longitudinal direction and the radial direction, and perpendicular to the z direction (defined below). Unless otherwise specified, references to “length” herein refer to a dimension along the longitudinal direction; references to “width” herein refer to a dimension along the circumferential direction.

With respect to a tampon and an applicator, the term “forward” refers to a longitudinal direction of movement during normal insertion of the tampon and/or applicator by a user, and refers to portions of the tampon or applicator that lie closer to and/or enter the vaginal cavity earlier than other portions during normal insertion. Conversely, the term “rearward” refers to a longitudinal direction of movement during normal withdrawal of the tampon and/or applicator by a user, and refers to portions of the tampon or applicator that lie farther from and/or exit the vaginal cavity earlier than other portions during normal withdrawal.

A “nonwoven,” “nonwoven web,” “nonwoven web material,” or “nonwoven fabric” is a cloth-like web material (or portion or section thereof) formed predominantly of fibers that are neither knitted nor woven, but rather, laid down and accumulated to a desired basis weight, then consolidated and held together to form a web, via one or any combination of calendering, thermal and/or compression bonding, bonding via use of a binder, heating (via, e.g., heated air driven through an accumulation of fibers) or hydroentangling (spunlace). The predominant fibers may be natural fibers harvested from plant material (e.g., cotton) (but excluding tree wood pulp), semi-synthetic (e.g., rayon, lyocell, viscose), or synthetic (e.g., fibers spun from molten thermoplastic polymer resin(s)), or any combination thereof. Herein, a skin- or membrane-like film (e.g., extruded or otherwise formed from polymer resin(s)) is not deemed a nonwoven. Herein, a paper tissue product, paper product, or paperboard or cardboard product, formed via wetlaying and predominantly constituted of tree wood pulp, is not deemed a nonwoven.

“Opened configuration,” with respect to a tampon, means the configuration of the pledget prior to the time it is compressed and formed into a self-sustaining form during manufacture, or in the case of a finished product, after it is completely ejected from an applicator (if present) and/or allowed and/or caused by any suitable technique to open and substantially re-assume its pre-compression shape and size.

“Paper” or “Cardboard” means a material in web or sheet form, formed predominantly of cellulose fibers, for example, wood pulp fibers, which have been suspended in a slurry, which is then poured onto a moving mesh belt, drained of water, and subsequently dried over drying rollers, and in many examples, finished via calendering. In the resulting web or sheet product the cellulose fibers are interlaid and randomly oriented. The paper or cardboard herein may also be substantially void of synthetic materials as defined herein, such as fibers, films, and/or adhesives. It is also to be appreciated that paper or cardboard may be formed by pulp molding.

“Predominant,” and forms thereof, when used to characterize a quantity of a constituent present in a composition, means that a majority of the weight of the material is constituted by the constituent.

“Withdrawal cord” refers to any section of string, yarn, cord, ribbon, strip material or other flexible/pliable elongate structure typically (although not necessarily) formed of fibrous material, attached to and/or extending from a tampon pledget and trailing from its rearward end. A withdrawal cord of sufficient length may be provided with a tampon for the purpose of providing a relatively thin and flexible trailing member of sufficient length to allow for a portion thereof to trail and remain outside of the introitus following full insertion of the tampon, which the user may easily grasp and pull to withdraw the tampon from her body following a desired duration of use.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

With respect to a paper sheet or web product, when laid out flat on a horizontal planar surface, the “z direction” is a direction orthogonal to the horizontal planar surface and is the direction along which caliper or thickness of the sheet or web (prior to rolling into paper tube product) would be measured.

“Synthetic materials” are those that are chemically modified such that the polymerization was not naturally occurring. Synthetic fibers, for example, include synthetic polymeric materials and bio-plastic polymers, such as polyethylene (PE), polyethylene terephthalate (PET), polylactic acid (PLA), polyhydroxyalkanoates, polybutylene succinate. Fibers that are not synthetic, for example, include cotton, flax, hemp, rayon, and jute. Substantially free of synthetic materials means that the article or component of the article is at least about 90% or at least 95% or at least about 98% by weight void of materials where the polymerization did not happen in nature or was not naturally occurring.

### Applicator Features

The present disclosure related to absorbent articles including a tampon, and more particularly, to a tampon having an outer member and an inner member such that the inner member is slidably engaged with the outer member. The outer member has an outer surface surrounding a longitudinal applicator axis. The outer member includes a barrel region and a grip region. The grip region includes greater than four circumferentially formed faces. These greater than four faces provide greater functionality and ease of use by providing multiple areas for the user to grip and securely hold the tampon during use. Each of the outer member and the inner member may include one or more layers that are spirally or convolutely wound to form the outer member and the inner member. Each of these one or more layers may comprise, consist essentially of, or consist of cellulosic material. Stated another way, each of the one or more layers may be void of synthetic fibers and films. A carboard applicator, as referred to herein, is an applicator that includes one or more layers and each of the one or more layers comprise, consists essentially of, or consists of cellulosic material and is void of synthetic fibers and films. “Synthetic” means that the polymerization did not happen in nature or was not naturally occurring. Synthetic materials are those that are chemically modified such that the polymerization was not naturally occurring. Synthetic fibers, for example, include synthetic polymeric materials and bio-plastic polymers, such as polyethylene (PE), polyethylene terephthalate (PET), polylactic acid (PLA), polyhydroxyalkanoates, polybutylene succinate. Materials that are not synthetic, for example, include cotton, flax, hemp, rayon, and jute. Free of synthetic materials means that the article or component of the article is at least about 90% or at least 95% or at least about 98% by weight void of materials where the polymerization did not happen in nature or was not naturally occurring.

Thus, the tampon of the present disclosure may have a cardboard applicator that provides greater functionality and convenience, and a more appealing ergonomic and aesthetic experience.

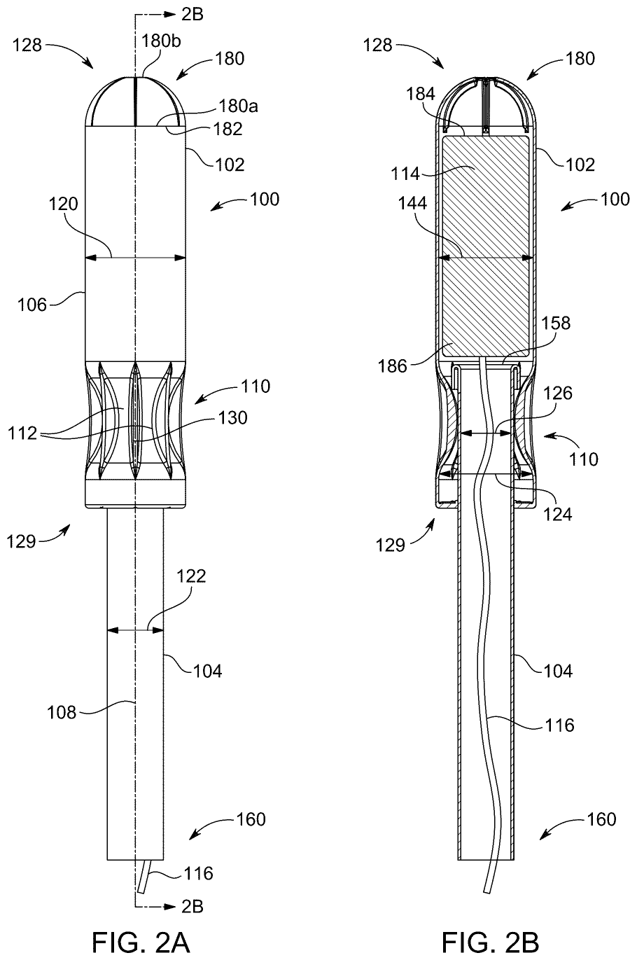

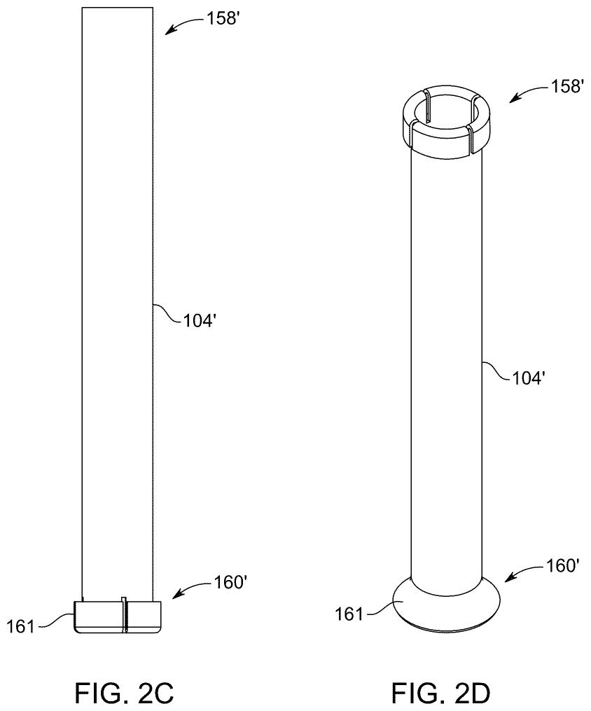

Referring to FIGS. 2A and 2B, a tampon in a self-sustaining form, including a pledget 114 having a forward end 184 and rearward end 186, and a withdrawal cord 116 attached to the pledget and trailing from the rearward end 186, may be supplied inside an applicator 100. Applicator 100 may be configured to house the tampon and protect it from contamination and/or unintended deformation prior to its use.

Applicator 100 may include an outer member including a hollow barrel region 102 and an ejection plunger 104 (also referred to herein as an inner member), which also may be hollow. The outer member, including the barrel region 102, and the ejection plunger 104 may be configured such that the ejection plunger 104 is at least partially disposed and longitudinally slidable within the barrel region 102. In some examples including the examples illustrated in FIGS. 2A and 2B, each of the barrel region 102 and the ejection plunger 104 are hollow approximately cylindrical bodies, wherein the cylindrical form of ejection plunger 104 has an external diameter 122 that is smaller than an inner diameter 144 of the barrel region 102, such that the barrel region 102 and the ejection plunger 104 may be concentrically arranged and longitudinally slidable with respect to each other in a telescoping manner. In some embodiments, the external diameter of the plunger or inner member is less than about 70% of the external diameter of the barrel.

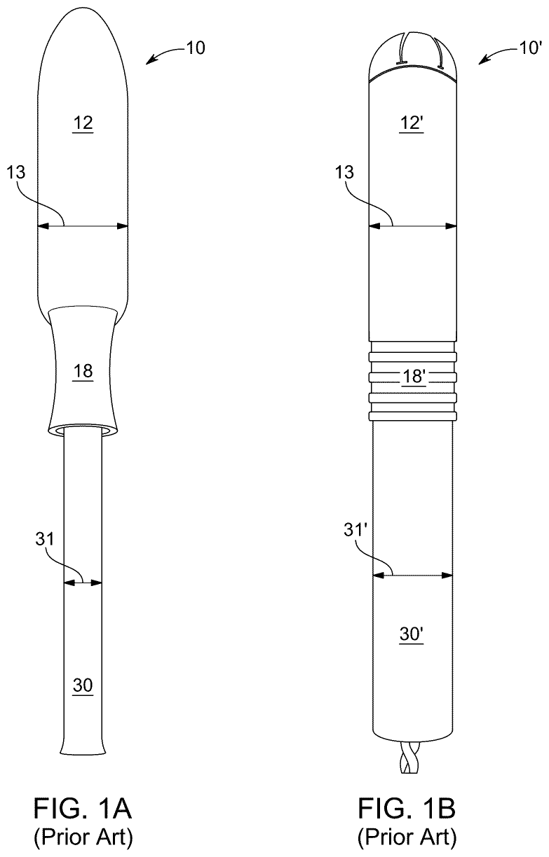

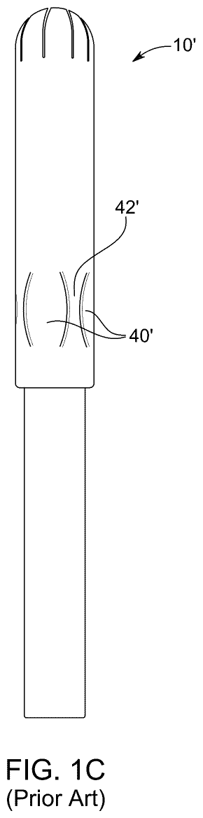

As shown in FIG. 2A, the outer member includes an outer surface 106 surrounding a longitudinal applicator axis 108, the barrel region 102, and the grip region 110. In some embodiments, the barrel region 102 has an external diameter 120 that is greater than the external diameter 122 of the plunger 104. In one example embodiment, the external diameter 120 of the barrel region 102 is about 16 mm or in a range from about 12 mm to about 20 mm and the external diameter 122 of the plunger 104 is about 7 mm or in a range from about 6 mm to about 8 mm. In some embodiments, the external diameter 122 of the plunger 104 is less than about 70% or less than about 60% or less than about 50% or less than about 40% of the external diameter 120 of the barrel region 102. Unlike the conventional cardboard applicator 10′ (FIG. 1B) where the external diameter 31′ of the plunger 30′ is about 80-90% of the external diameter 13′ of the barrel region 12′, the external diameter 122 of the plunger 104 is about 50%-60% of the external diameter 120 of the barrel region 102. The inventors of the present disclosure recognized that the external diameter 31′ of the plunger 30′ in the conventional cardboard applicator 10′ had to be at least 80-90% of the external diameter 13′ of the barrel region 12′ to avoid the plunger 30′ wobbling as it is slid into or withdrawn from the barrel region 12′. The improved cardboard applicator 100 herein discloses an improved grip region 110 to accommodate the external diameter 122 of the plunger 104 only being 50-60% of the external diameter 120 of the barrel region 102 in order to prevent the undesired wobbling between the inner member 104 and outer member.

The outer member may be provided with additional functional features. For example, a forward end 128 of the outer member may include an insertion end region adjacent to the barrel region. The insertion end region includes a plurality of petals 180 that have proximal bases 180a having outer surfaces coincident with the general cylindrical shape of the barrel region 102, and distal ends 180b disposed radially inwardly of the proximal portions. The forward end 128 of the barrel region 102 is opposite from the grip region 110. The petals 180 and/or remainder of the barrel region 102 may be configured such that the petals 180 may flex about the proximal bases 180a such that, upon forward longitudinal pressure exerted by the forward end 184 of the pledget 114 during ejection via pressure form longitudinal forward movement of the ejection plunger 104 relative to the barrel region 102, the petals 180 will flex outwardly about their bases. This expands an opening at the forward end 128 of the barrel region 102, which allows the pledget 114 to pass forwardly through the forward end 128 of the barrel region 102, and out of the applicator 100. The petals 180 may be integrally formed with the remainder of the barrel region 102. In some examples, the petals may be formed by making cuts in a forward portion of a cylindrical precursor body of the barrel region 102, and then plastically deforming the remaining uncut portions radially inwardly, and with suitable curvature. Such deformation may be accomplished in some examples via use of a forming die or mold (not shown), and may be facilitated in some examples via application of heat and/or steam. Additionally, in some examples the barrel region 102 may be imparted with localized flexure zones 182 that demark the proximal bases 180a of the petals, which are zones about which the material of the barrel region 102 and/or petals more readily flexes or hinges, for example, as a result of a localized reduction in thickness or caliper of the material in flexure zones 182. Flexure zones 182 may be imparted by, e.g., molding or localized compression applied during the manufacturing process.

The outer member may also be provided with a grip region 110 having gripping surface features (e.g., circumferential faces 112) that enable the user to tactilely identify a rearward end region 129 of the outer member, and also enhance gripping slip resistance during use of the applicator 100. In a simple form, such as shown in FIGS. 2A and 2B, the features of the grip region 110 may be a series of circumferential faces 112 spaced apart by respective ribs 130 about the outer surface 106 of the outer member proximate its rearward end 129. More complex gripping surface features may be provided, however, of any design desired for purposes of functionality and esthetic appeal. Gripping surface features may be imparted to outer member via forming techniques including molding, embossing, etc.

As illustrated in FIG. 2B, the barrel region 102 may be sized to house the tampon pledget 114 in its self-sustaining form. It may be desired that the barrel region 102 have an internal diameter 144 that is small enough relative the outer lateral dimension of the pledget 114 such that the pledget 114 cannot freely slide longitudinally within the barrel region 102 (due to slight interference or radial compression and light static friction resistance), but not so small as to create unacceptable static friction resistance and thereby require the user to exert unacceptably great force on the ejection plunger or inner member 104 to eject the pledget 114 from the outer member, when ejection is desired.

Also as suggested in FIG. 2A, it may be desired that the ejection plunger 104 have the external diameter 122 (of its cylindrical form) that is equal to or smaller than the internal diameter 144 or other inner lateral dimension of the grip region. It may be desired to provide such clearance within 1% of parity such that the ejection plunger 104 cannot freely and unintentionally slide coaxially/longitudinally within or out of the barrel region 102 in the absence of intentionally applied force, but such that the force required to slide the ejection plunger 104 within the grip region is not so great as to be unacceptably great to the user, or even potentially destructive to the applicator.

Referring to FIGS. 2A and 2B, the ejection plunger 104 may be provided with an inner diameter that is smaller than an outer diameter or lateral dimension of the tampon pledget 114 (or other object) housed within the barrel region 102. With this size relationship, longitudinal force applied to the ejection plunger 104 urging it into the barrel region 102 will cause its forward end or first end region 158 to contact the rearward end 186 of the tampon pledget 114 (or other object) and urge the object forward and out the forward end 128 of the barrel region 102. As an alternative, or in addition to, such inner diameter size at the forward end region or first end region 158 of the ejection plunger 104 may have its circumferential edge rolled over inwardly or otherwise formed to extend radially inwardly, thereby effectively reducing the inner diameter or inner lateral dimension of the ejection plunger 104 at its forward end 158. The first end region 158 of the ejection plunger or inner member 104 may be outwardly rolled or flanged to increase the amount of area at the forward end 158 of the ejection plunger 104 that effectively contacts the rearward end 186 of the pledget 114 or other object within the barrel region 102, to transmit ejection force exerted by the user. A similar feature can be imparted to the rearward end or second end region 160 of the ejection plunger 104, to expand contact surface area and decrease focused/localized pressure on the user's finger at the circumferential rim of the rearward end 160 when the user applies ejection force at the rearward end 160, and thereby make application of ejection force more comfortable to the user's finger. In some embodiments, the pledget 114 is configured with an expulsion force of less than less than 900 gram-force (gf) or less than 800 gram-force (gf) or in a range of about 400-450 gf or from about 250 gf and about 700 gf.

In some embodiments, the barrel region 102 is manufactured such that the internal diameter 144 thereof is less than about 19 mm and preferably less than about 16 mm. Although the external diameter of typical menstrual-use tampons varies, most menstrual-use tampons currently marketed have an external diameter of less than about 19 mm. However, if one desires to use the applicator of the present disclosure for purposes other than delivery of a menstrual-use tampon to a human female vaginal cavity (such as, for example, delivery of other devices, medications, etc. to other cavities in humans, or cavities in animals (veterinary use)), a barrel region 102 with a larger diameter may be desired.

The material of the outer member may be overlapped into a tubular configuration. Spirally or convolutely winding the outer member into a cylindrical tube is especially advantageous when the outer member is formed from a laminate. The reason for this is that when a laminate is circumferentially wound into a tube and a butt seam or an overlap is formed, a common problem with a rigid or stiff walled, tubular member having a relatively small diameter and a butt seam is that the seam may tend to come apart after formation if exposed to certain stress forces and/or high humidity. Accordingly, it may be preferred that the barrel region 102 be formed into a cylindrical configuration without the presence of a butt seam or an overlap.

The ejection plunger 104, also referred to herein as an inner member, may be manufactured of similar materials and in a similar layered and wound configuration as the outer member, with appropriate adjustments to its size (length and outer diameter) to enable it to fit within the grip region 110 with appropriate clearance, and have sufficient length to facilitate full ejection of the tampon pledget (or other object) from the barrel region 102.

For a menstrual-use tampon, the barrel region 102 may be sized and configured to snugly house the tampon. As noted above, it may be desired that the outer member have a relatively smooth exterior surface that will facilitate comfortable insertion of the barrel region 102 into the vaginal cavity. When the exterior surface is smooth (and preferably has surface properties and/or surface treatment that provide appropriately low friction with sensitive tissue), the barrel region 102 will easily slide within the vaginal cavity without subjecting the internal tissues of the vagina to irritation or abrasion. The exterior surface of the barrel region 102 may be coated to give it appropriate low friction characteristic surface properties. Wax, polyethylene, a combination of wax and polyethylene, and cellophane are representative components of coatings that have been applied to barrel regions of tampon applicators, to facilitate comfortable use.

The applicator of the present disclosure may be used for the delivery of menstrual-use devices, such as a tampon, an intravaginal collection device (e.g., menstrual cup), and interlabial pads. The applicator of the present disclosure may be used for the delivery of a pessary. The applicator may also be useful for delivery of oral, rectal, and vaginal suppositories, as well as nasal devices, such as nasal tampons. The applicator may be used for delivery of various other materials including, medicaments, moisturizers, vitamins and minerals, spermicides, and odor controlling agents. These materials may be delivered in the form of rigid solid objects, deformable solid objects, creams, foams, gels, etc. The applicator may be adapted for human or animal/veterinary use.

FIG. 2B depicts a non-limiting example of a tampon having the pledget 114 in a self-sustaining form and having a forward end 184 and a rearward end 186 and a withdrawal cord 116 attached to the pledget 114 and extending rearward from a location proximate the rearward end 186 of the pledget 114. Generally, the “pledget” as referred to herein is that portion of the tampon that includes the main body and mass of absorbent material, but does not include withdrawal cord 116. Tampons contemplated herein, however, are not limited to structures having the particular configuration shown in the drawing. In some embodiments, at least a portion of the cord 116 is braided. In other embodiments, there may be two different portions of the cord 116 that are each braided. In this example embodiment, one of the braided portions is a thick portion and the other of the braided portions is a thinner portion, as disclosed in U.S. Patent Publication No. 2023/0058874 that is incorporated by reference herein.

As shown in FIG. 2B, the pledget 114 of the tampon has the forward end 184 and the rearward end 186. During manufacture of the tampons the pledget 114 may be folded, bunched, compressed and/or otherwise formed in size and shape, from its initially manufactured configuration into a generally cylindrical and/or capsule-shaped configuration (e.g., as shown in FIG. 2B) along a radial direction, the lateral direction, longitudinal direction, or in some combination thereof.

FIG. 2C is a longitudinal side view of a plunger 104′ (also referred to herein as an inner member) of the tampon product of FIG. 2A with a flange 161 at a second end region 160′ thereof. During use of the applicator 100, the plunger 104′ includes the forward end 158 and the rearward end 160 opposite to the forward end 158. The forward end 158 of the plunger 104′ is configured to engage a portion of the pledget 114 and the rearward end 160′ includes the flange 161. In some embodiments, the flange 161 can have a diameter greater than the internal diameter of the barrel region 102 at the rearward end 129 or the flange can have a diameter greater than a portion of the internal diameter of the grip region 110, to keep the plunger 104′ from unintentionally going through the barrel region 102 and out the forward end of the outer member, and to provide a surface on which the consumer or user can push to expel the pledget 114 from the forward end 128 of the barrel region 102, such as shown on FIG. 2D. In some embodiments, the flange 161 may be a pushed-out petal, a compressed portion, an embossed portion, or added material to increase the diameter of the plunger 104′ at the rearward end 160′. It is also to be appreciated that each of the first end region and/or the second end region may include a portion that has been rolled inwardly or outwardly or flanged.

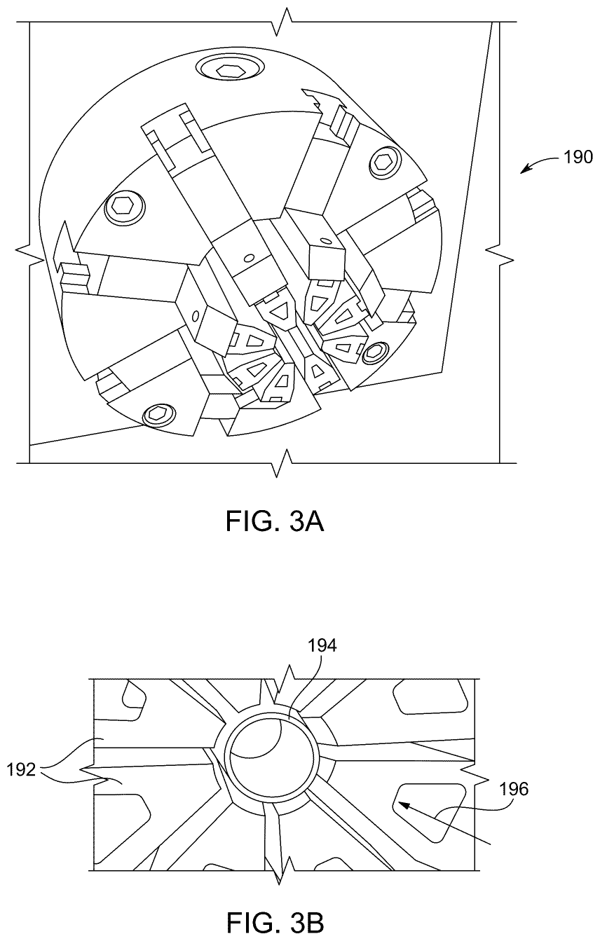

FIG. 3A is a perspective view of a compression device 190 used to form circumferential faces 112 in the grip region 110 of the applicator 100 of FIG. 2A. FIG. 3B is a perspective view of a plurality of forming members 192 of the machine 190 of FIG. 3A that move radially inward to form the circumferential faces 112 in the grip region 110 of the applicator 100. As shown in FIG. 3B to form the grip region 110 in the applicator 100, a tube 194, which may be a cardboard or paper tube, is positioned within an opening of the compression device 190. In some embodiments, the tube 194 is a spirally wound group of one or more layers, at least one of which includes cellulosic material. In other embodiments, the tube 194 is convolutely wound layers of material including at least one layer of cellulosic material.

In use, after positioning the paper or cardboard tube 194 in the opening defined by the compression device 190, the forming members 192 are moved in an inward radial direction 196 and engage the outer surface of the tube 194 to form the circumferential faces 112 and ribs 130 therebetween. After forming the circumferential faces 112, the forming members 192 are moved in an opposite radial direction. The tube 194 is then moved or advanced such that a different section of the tube 194 is positioned within the opening and the process is repeated so that the grip region 110 including the circumferential faces 112 are formed at a plurality of spaced apart regions along the tube 194. The tube 194 is then removed from the compression device 190 and cut into individual parts where each part is used to form the barrel region 102 of the applicator 100. In other embodiments, the tube 194 may be first cut into a plurality of parts and each respective part is positioned in the opening of the compression device 190 and the forming members 192 are used to form the circumferential faces 112 in each part of the tube 194. The forming members 192 may be used to form the circumferential faces 112 without using a mandrel, a spindle, or any supporting material or device positioned inside the tube 194.

The inventors of the present invention recognized that the compression device 190 should be operated such that the paper or cardboard tube 194 is compressed to form the circumferential faces 112 without forming slits or holes in the tube 194. The inventors of the present disclosure surprisingly found that the tube 194 material can be compressed to form the circumferential faces 112 without affecting the color of the tube 194. In some example embodiments, the forming members 192 of the compression device 190 radially compress the tube 194 material and pinch the material between two adjacent forming members 192 to form the circumferential faces 112 with ribs 130 therebetween. In some embodiments, heat is used in conjunction with the compression and squeezing of the forming members 192 but in other embodiments no heat is employed. The inventors of the present disclosure noted that there is some rebound to the tube 194 material as it is compressed to form the circumferential faces 112 and ribs 130.

The inventors of the present disclosure noted that for the conventional cardboard applicator 10′ (FIG. 1C), the greatest number of circumferential faces formed in the cardboard material is four. Thus, the inventors of the present disclosure herein configured the compression device 190 to form greater than four circumferential faces 112 in the grip region 110. This is due to a larger number of circumferential faces in the grip region enhancing the control of the user over the barrel region 102 and/or the inner member 104. Unlike plastic material, which is relatively easy to mold and shape, the inventors of the present disclosure recognized that cardboard material is more challenging to mold and shape and thus had to overcome design obstacles not present in forming plastic applicators. For example, paper or carboard is prone to tearing when too much pressure is applied, and paper or cardboard tends to fold and produce wrinkles that would likely be aesthetically displeasing to consumers.

As previously discussed, conventional cardboard applicators 10′ (FIG. 1B) feature the barrel region 12′ with the external barrel diameter 13′ that is relatively close to the external plunger diameter 31′ of the plunger 30′. The inventors of the present disclosure expected that the compression and pinching of the paper or cardboard tube 194 with the forming members 192 would cause undesirable fold lines in the tube 194. Instead, the inventors of the present disclosure surprisingly found that the tube 194 tolerates the compression and squeezing of the forming members 192 relatively well, resulting in a surprisingly smooth surface to the circumferential faces 112 and ribs 130. Additionally, the inventors of the present disclosure expected that the compression and pinching of the tube 194 by the forming members 192 would require an additional heating step to form the circumferential faces 112 and ribs 130. However, the inventors surprisingly found that no additional heating step was required.

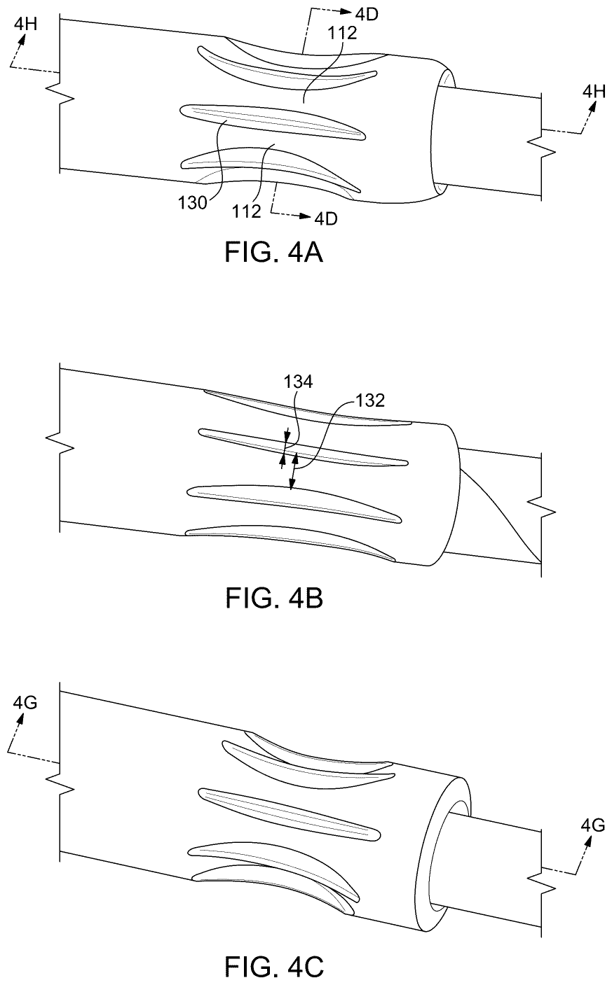

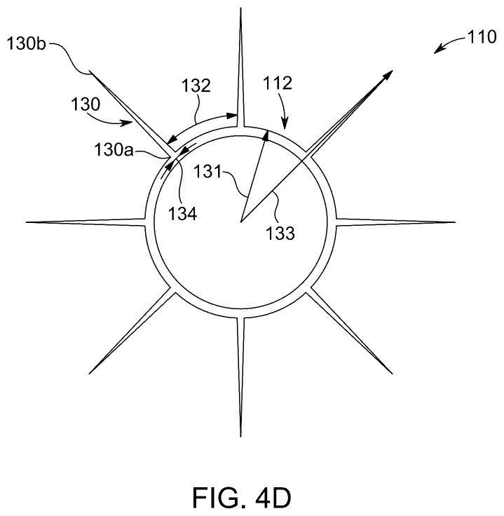

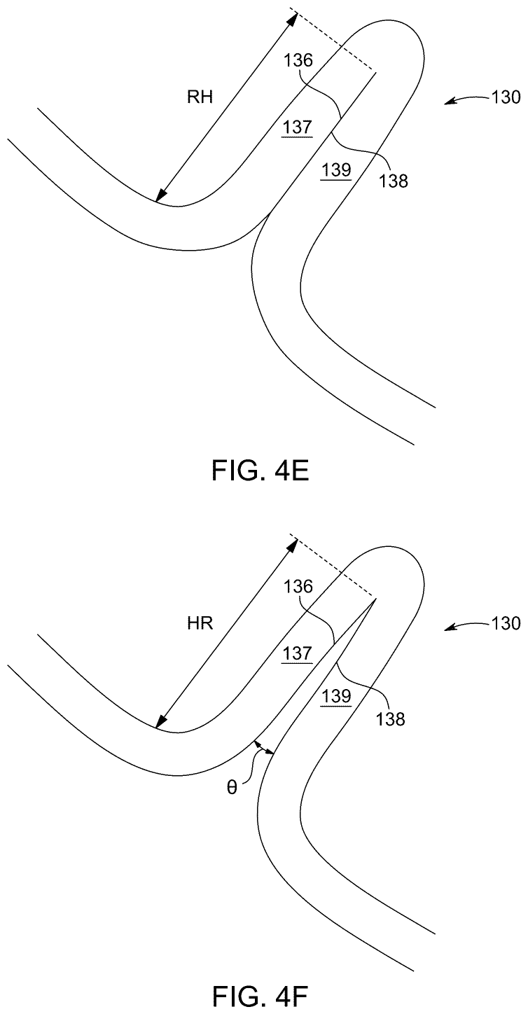

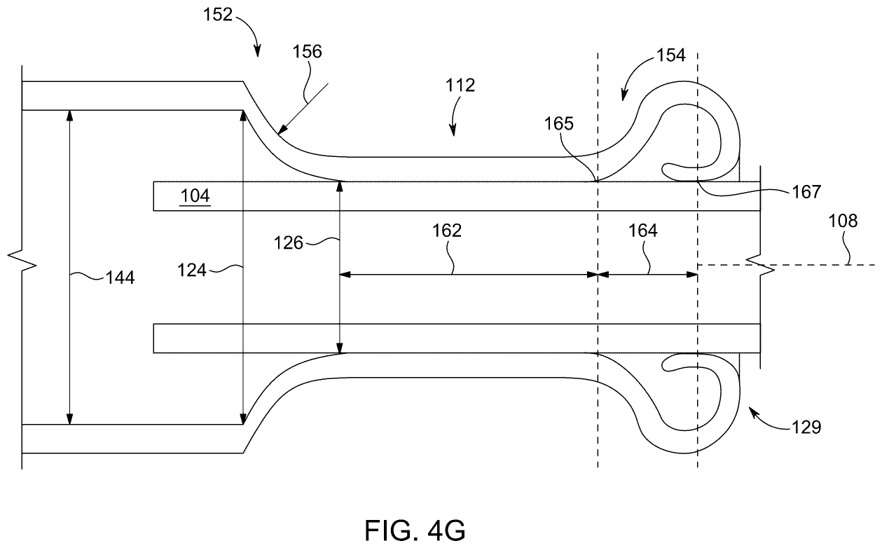

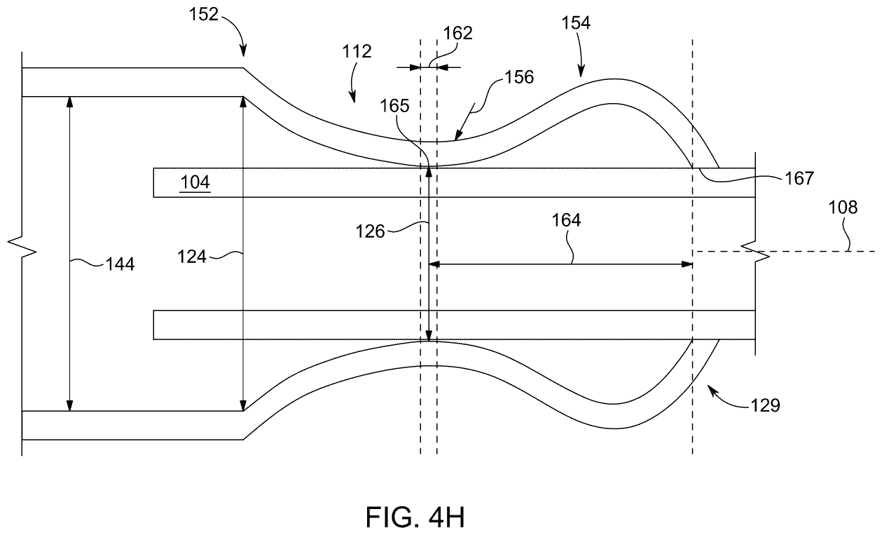

FIGS. 4A through 4C are perspective and top views of a plurality of circumferential faces 112 and ribs 130 therebetween of the grip region 110 of the applicator of FIG. 2A. In some embodiments, the circumferential faces 112 and ribs 130 therebetween are formed using the compression device 190 of FIGS. 3A and 3B. The circumferential faces may extend from an upper grip region to a lower grip region, as shown in FIGS. 4A-4C. Each of the faces 112 of the grip region 110, at least over a portion of the grip region 110, may be at least one of concave and convex. For example, two or more of the faces 112 may include a first concave region, a substantially planar region, and a second concave region. In another example, two or more of the faces 112 may be at least one of concave and convex extending from the upper grip region to the lower grip region. Each of the faces 112 may be identical in terms of shape and/or dimension. Each face 112 may have one or more of a same width (measured perpendicular to the longitudinal applicator axis 108), a same length (measured along the longitudinal applicator axis 108) and a same shape (e.g., same convex or concave structure). In these embodiments, when operating the compression device 190 of FIGS. 3A and 3B, the compression by the forming members 192 is symmetrical in the inner radial direction 196 and/or each chuck 192 has a same shape or curvature to thereby compress a same-shaped circumferential face 112. In some embodiments, if an odd number of circumferential faces 112 are to be formed in the cardboard tube 194 then the compression force of the forming members 192 are to be symmetrical. In some embodiments, if an even number of circumferential faces 112 are to be formed in the cardboard tube 194 then faces which are radially opposite from each other have the same shape (e.g., be compressed by forming members 192 having a same shape).

As further shown in FIGS. 4A through 4C, in some embodiments the grip region 110 includes greater than four circumferentially formed faces 114. In other embodiments, the grip region 110 includes greater than or equal to six circumferential faces 114. In still other embodiments, the grip region 110 includes greater than or equal to eight circumferentially formed faces 114. As further shown in FIGS. 4A through 4C, the grip region 110 is continuously circumferential so that there are no slits or openings in the grip region 110. As further shown in FIGS. 4A through 4C, in some embodiments the grip region 110 comprises greater than four outwardly projecting ribs 130, where each rib 130 is positioned between two adjacent circumferential faces 112. The inventors of the present disclosure recognized that an increased number of circumferential faces enhance the user ability to grip the grip region 110. The peak region of the rib may be substantially planar with the external surface of the barrel region, or the peak region of the rib may be concave and/or convex with respect to the external surface of the barrel region. If the peak region of the rib is convex, the convexity of the peak region of the rib is less than the convexity of the face. Stated another way, the radius of curvature of the peak region of the rib is different and/or greater than the radius of curvature of the face.

In some embodiments, the circumferential faces 112 of the grip region 110 include one or more indicia, such as printed indicia and/or tactile indicia. Such tactile indicia may feature embossed designs that may be formed through the same process used to form the circumferential faces using the compression device or in a subsequent or proceeding separate process. In still other example embodiments, additional material may be included outside of the grip region 110. Such additional material may include printed material (e.g., graphics, alphanumeric characters, etc.) or a material additive to create texture (tactile indica) in the grip region 110. In some embodiments, a first face comprises a first indicia and a second face comprises a second indicia. The first indica and the second indica may be the same or different. Further, the first indicia and the second indicia may be printed indicia and/or tactile indica.

FIG. 4D is a cross-sectional view of the grip region 110 of FIG. 4A taken along the line 4D-4D. As shown in FIGS. 4B and 4D, in some embodiments each of the circumferential faces 112 has a face circumferential width 132 and each of the ribs 130 has a rib circumferential width 134. In some embodiments, the face circumferential width 132 is greater than the rib circumferential width 134. In some embodiments, each of the faces 112 has the same face circumferential width 132 and each of the ribs 130 has the same rib circumferential width 134. The rib circumferential width 134 is measured at a rib base region 130a of the rib 130 (e.g., where the rib 130 intersects the face 112). The face circumferential width 132 is measured adjacent the face 112 surface. In some embodiments, the face circumferential width 132 is about 2 mm or in a range from about 0.03 mm to about 4.1 mm and the rib circumferential width 134 is about 0.6 mm or in a range from about 0.4 mm to about 0.8 mm. Thus, in these embodiments, a ratio of the face circumferential width 132 to the rib circumferential width 134 is about 3.0 or about 4.0 or in a range from about 0.1 to about 10.0 or from about 2.0 to about 10.0 or from about 3.0 to about 10.0. Thus, this design of the grip region 110 is distinct from the conventional cardboard applicator 10′ (FIG. 1C) with about a 1:1 ratio between the face circumferential width and the rib circumferential width. The inventors recognized that this alternate ratio in the disclosed cardboard applicator 100 enhances the grip of the user on the grip region and thus advantageously enhances the user control of the applicator during use. The rib circumferential width 134 may be variable from the rib base region 130a to the rib peak region 130b. Stated another way, the rib 130 may be tapered from the rib base region 130a to the rib peak region 130b such that the rib base region 130a has a greater width than the rib peak region 130b. In some embodiments, the rib circumferential width 134 may be substantially the same in the rib peak region 130b and the rib base region 130a.

In some other embodiments, such as shown in FIG. 4D each of the circumferential faces 112 has an external face radius 131 and each of the ribs 130 has external rib radius 133. Along at least a portion of the face, the external rib radius 133 is greater than the external face radius 131. Each of the faces 112 may have the same external face radius 131 and each of the ribs 130 may have the same external rib radius 133. In some embodiments, the external rib radius 133 is about 7.0 mm or in a range from about 6.0 mm to about 10.0 mm and the external face radius 131 is about 3.5 mm or in a range from about 3.0 mm to about 7.0 mm. Thus, in these embodiments, a ratio of the external rib radius 133 to the external face radius 131 is about 2.0 or in a range from about 0.8 to about 3.0. Thus, this is distinct from the conventional cardboard applicator 10′ (FIG. 1C) where the ratio is much smaller which reduces the ability of the user to engage and grip the grip region of the applicator. The inventors recognized that this larger ratio in the disclosed cardboard applicator 100 enhances the grip of the user on the grip region (e.g., by providing sufficient radial space to position a user's finger so to grip one or more ribs 130) and thus advantageously enhances the user control of the applicator during use. In some embodiments, a distance between the peak region 130b of a first rib 130 and the peak region 130b of a second rib 130 that is radially opposite of the first rib 130 is about 17 mm or in a range from about 15 mm to about 20 mm.

FIG. 4E is a cross-sectional view of a rib of the cross-sectional view of the grip region 110 of FIG. 4A. As shown in FIG. 4E, in some embodiments, the rib 130 includes a first rib portion 137 and a second rib portion 139 that are compressed or squeezed together (e.g., by the forming members 192 of the compression device 190) to form the rib 130. In some embodiments, a first internal rib surface 136 of the first rib portion 137 is in contact with a second internal rib surface 138 of the second rib portion 137. In some embodiments, the first internal rib surface 136 contacts the second internal rib surface 138 over at least a portion (e.g., greater than about 5% or greater than about 10% or greater than about 15%) of a rib height RH of the rib 130. In some embodiments, the first internal rib surface 136 contacts the second internal rib surface 138 over at least a majority (e.g., greater than 50%) of a height RH of the rib 130. In other embodiments, the first internal rib surface 136 contacts the second internal rib surface 138 over the entire height RH of the rib 130. The inventors of the present disclosure recognized that having these internal rib surfaces 136, 138 contact each other over at least a portion of the rib 130 height maximizes the face circumferential width 132 and/or minimizes the rib circumferential width 134. Consequently, as previously discussed, this advantageously enhances the user control over the grip region 110 by enhancing the ability of the user to grip the barrel region 102.

FIG. 4F is a cross-sectional view of a rib of the cross-sectional view of the grip region 110. As shown in FIG. 4F, in some embodiments, the rib 130 includes a first rib portion 137 and a second rib portion 139 that are compressed or squeezed together (e.g., by the forming members 192 of the compression device 190) to form the rib 130. In some embodiments, a first internal rib surface 136 of the first rib portion 137 is in separated from a second internal rib surface 138 of the second rib portion 137 by a rib angle Q. The rib angle θ may be from about 0 degrees to about 15 degrees or from about 0 degrees to about 10 degrees, including all 0.1 degree increments form therein and thereby the recited range. The inventors of the present disclosure recognized that minimizing the distance between the internal rib surfaces 136, 138 maximizes the face circumferential width 132 and/or minimizes the rib circumferential width 134. Stated another way, the inventors of the present disclosure recognized that angling the internal rib surfaces 136, 138 with respect to one another maximizes the face circumferential width 132 and/or minimizes the rib circumferential width 134. Consequently, as previously discussed, this advantageously enhances the user control over the grip region 110 by enhancing the ability of the user to grip the barrel region 102. The amount of rebound or separation between the first internal rib surface and the second internal rib surface will be dependent on the aggressiveness of the amount of compression or the intended design, such as the ratio of rib radius to rib face radius. The larger the difference between these radii, the more likely there is to be a gap since the stress is concentrated on the tips of the rib and the fold at the base of the rib, causing the rib base to separate.

FIG. 4G is a cross-sectional view of the grip region 110 of FIG. 4B taken along the line 4G-4G. As shown in FIG. 4G, in some embodiments each of the circumferential faces 112 of the grip region 110 project inwardly toward the longitudinal applicator axis 108.

As further shown in FIG. 4G, the grip region 110 has a first internal grip diameter 124 and a second internal grip diameter 126, where the first internal grip diameter 124 is greater than the second internal grip diameter 126. Thus, in these embodiments, the grip region 110 features different diameters at different portions along the longitudinal applicator axis 108. In some embodiments, the first internal grip diameter 124 is about 13.3 mm or in a range from about 9.0 mm to about 20.0 mm and the second internal grip diameter 126 is about 6.0 mm or in a range from about 5.0 mm to about 14.0 mm. In some embodiments, a ratio of the second internal grip diameter 126 to the first internal grip diameter 124 is less than about 90%. The difference between the first internal grip diameter 124 and the second internal grip diameter 126 may be at least about 1 mm or at least about 1.5 mm or at least about 2 mm or at least about 2.5 mm or at least about 3 mm or at least about 3.5 mm or at least about 4 mm or at least about 5 mm or from about 1.5 mm to about 13 mm or from about 2.5 mm to about 10 mm. Thus, the circumferential faces 112 extend within the interior of the barrel region 102 towards the longitudinal applicator axis 108 by a greater extent than the faces 40′ in the conventional cardboard applicator 10′ (FIG. 1C). The inventors of the present disclosure recognized that this advantageously ensures tight engagement between the plunger 104 and the grip region 110. Since the disclosed applicator 100 herein features the external plunger diameter 122 being noticeably less than the external barrel diameter 120 (FIG. 2A). The external plunger diameter 122 is measured (or defined) where the flange 161 or marking in regions 170, 172, 174 are not present. The inventors of the present disclosure recognized that this ratio between the second internal grip diameter 126 and first internal grip diameter 124 facilitates tight engagement between the plunger 104 and the barrel region 102. In some embodiments, the second internal grip diameter 126 is at least 30% of the first internal grip diameter 124. In some embodiments, a difference between the first internal grip diameter 124 and the second internal grip diameter 126 is at least about 1 mm and/or about 2.5 mm and/or about 3 mm and/or about 3.5 mm and/or about 4 mm or from about 1 mm to about 15 mm.

The inventors of the present disclosure designed the grip region 110 so that the barrel region 102 may support the plunger 104 at multiple regions along the longitudinal applicator axis 108. As shown in FIG. 4G, in some embodiments the plunger 104 is supported by the grip region 110 (at the second internal grip diameter 126) and is also supported at the rearward end 129 of the barrel region 102. As illustrated in FIGS. 4C and 4G, the rearward end region 129 of may be inverted or rolled such that an external surface of the rearward end is configured to become an internal surface and engage a portion of the plunger 104. The external plunger diameter 122 of the plunger 104 (FIG. 2A) is greater than 90% of the second internal grip diameter 126 of the grip region 110. The inventors of the present disclosure recognized that this facilitates tight engagement between the plunger 104 and the grip region 110 while still permitting the plunger 104 to slide within the barrel region 102 relative to the grip region 110. For purposes of this disclosure, “tight engagement” corresponds to a tolerance (e.g., difference between the external plunger diameter 122 and the second internal grip diameter 126) that is about 0.3 mm to about 0.5 mm and/or about 0.2 mm to about 0.6 mm and/or about 0.1 mm to about 0.8 mm.

As shown in FIG. 4G, in some embodiments the second internal grip diameter 126 of the grip region 110 contacts the plunger 104 over a minimum longitudinal length 162 along the longitudinal applicator axis 108. The minimum longitudinal length 162 may be at least 12 mm or at least about 15 mm or in a range from about 12 mm to about 20 mm along the longitudinal applicator axis 108. As shown in FIG. 4G in some embodiments the circumferential faces 112 of the grip region 110 are substantially flat over the minimum longitudinal length 162.

As further shown in FIG. 4G, in some embodiments the grip region 110 of the barrel region 102 contacts the plunger 104 at a first contact point 165 and the rearward end 129 of the barrel region 102 contacts the plunger 104 at a second contact point 167. The contact points 165, 167 are spaced apart along the longitudinal applicator axis 108 by a distance 164. The distance 164 is about 8 mm or in a range from about 6 mm to about 10 mm. The inventors of the present disclosure recognized that providing multiple contact points 165, 167 between the plunger 104 and the barrel region 102 advantageously helps to stabilize the plunger 104 as it is inserted into and retracted from the barrel region 102. The inventors of the present disclosure also recognized that spacing the contact points 165, 167 by a distance disclosed herein further facilitates this stabilization.

As further shown in FIG. 4G, in some embodiments each of the circumferential faces 112 extends from an upper grip region 152 to a lower grip region 154. In some embodiments, the faces 112 have a radius of curvature 156 in one or both of the regions 152, 154 that is about 8.3 mm or in a range from about 0.1 mm to about 425 mm. The radius of curvature may be determined by taking the (face length)²/(face depth). The face length may be from about 3 m to about 30 mm and the face depth may be from about 0.1 mm to about 3 mm. For example, the face length may be about 25 mm and the face depth may be about 2 mm. In some embodiments, the radii of curvature is the same for each of the circumferential faces 112. In this example embodiment, the radius of curvature 156 is measured in a plane perpendicular to the longitudinal applicator axis 108. The inventors of the present disclosure recognized that having the radius of curvature 156 in this range advantageously maximizes the length of the circumferential faces 112 (e.g., along the longitudinal applicator axis 108) which further enhances the user ability to grip the grip region 110 and thus maximizes user control of the barrel region 102. Although FIG. 4G depicts that the grip region 110 is substantially cylindrical between the regions 152, 154 in other embodiments the grip region 110 is concave or convex between the regions 152, 154.

It is also to be appreciated that the rearward end region may not contact the plunger. The rearward end region may be shaped such that it extends outward away from the plunger, or stated another way, there is a gap between the plunger and the rearward end region. In those embodiments where the reward end region does not contact the plunger, the face may engage or contact the plunger along the minimum longitudinal length, as previously discussed.

As illustrated in FIG. 4H, in some embodiments the plunger 104 is supported by the grip region 110 (at the second internal grip diameter 126) and is also supported at the rearward end 129 of the outer member. As illustrated in FIGS. 4A and 4H, the rearward end region 129 of the barrel region 102 may be configured to engage a portion of the plunger 104. The external plunger diameter 122 of the plunger 104 (FIG. 2A) is greater than 90% of the second internal grip diameter 126 of the grip region 110. The inventors of the present disclosure recognized that this facilitates tight engagement between the plunger 104 and the grip region 110 while still permitting the plunger 104 to slide within the barrel region 102 relative to the grip region 110.

As shown in FIG. 4H, the second internal grip diameter 126 of the grip region 110 contacts the plunger 104 over a minimum longitudinal length 162 along the longitudinal applicator axis 108. The minimum longitudinal length 162 may be at least about 2 mm or at least about 5 mm or at least about 7 mm or in a range from about 2 mm to about 10 mm along the longitudinal applicator axis 108. As shown in FIG. 4H, in some embodiments, the circumferential faces 112 of the grip region 110 are substantially curved over the minimum longitudinal length 162.

As further shown in FIG. 4G, in some embodiments the grip region 110 of the barrel region 102 contacts the plunger 104 at a first contact point 165 and the rearward end 129 of the barrel region 102 contacts the plunger 104 at a second contact point 167. The contact points 165, 167 are spaced apart along the longitudinal applicator axis 108 by a distance 164. The distance 164 is about 8 mm or in a range from about 6 mm to about 10 mm. The inventors of the present disclosure recognized that providing multiple contact points 165, 167 between the plunger 104 and the barrel region 102 advantageously helps to stabilize the plunger 104 as it is inserted into and retracted from the barrel region 102. The inventors of the present disclosure also recognized that spacing the contact points 165, 167 by a distance disclosed herein further facilitates this stabilization.

As further shown in FIG. 4H, each of the circumferential faces 112 extends from an upper grip region 152 to a lower grip region 154. In some embodiments, the faces 112 have a radius of curvature 156 in one or both of the regions 152, 154 that is about 300 mm or in a range from about 4.3 mm to about 900 mm. The radius of curvature is determined by taking the (face length) 2/(face depth). The face length may be from about 3 m to about 30 mm and the face depth may be from about 0.1 mm to about 3 mm. For example, the face length may be about 25 mm and the face depth may be about 2 mm. In some embodiments, the radii of curvature is the same for each of the circumferential faces 112. The radius of curvature 156 is measured in a plane perpendicular to the longitudinal applicator axis 108. The inventors of the present disclosure recognized that having the radius of curvature 156 in this range advantageously maximizes the length of the circumferential faces 112 (e.g., along the longitudinal applicator axis 108) which further enhances the user ability to grip the grip region 110 and thus maximizes user control of the barrel region 102. Although FIG. 4H depicts that the grip region 110 is substantially concave between the regions 152, 154, the grip region 110 be substantially flat, concave, and/or convex between the regions 152, 154.

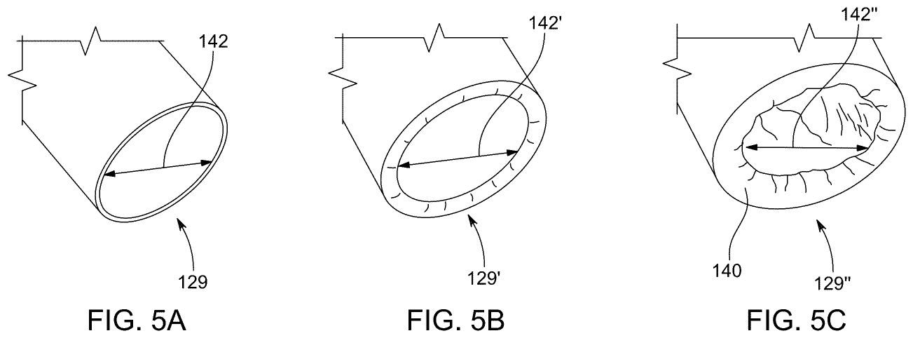

FIGS. 5A through 5C are perspective views of the rearward end 129 of the barrel region 102 of the applicator 100 of FIG. 2A. As shown in FIGS. 5A through 5C, in some embodiments the barrel region 102 includes a rearward end 129 disposed adjacent to the grip region 110. The rearward end 129 comprises an internal end region diameter 142. FIG. 5A shows the rearward end 129 with the internal end region diameter 142 without any modification to the rearward end 129. FIGS. 5B and 5C depict respective rearward ends 129′, 129″ where the rearward end has been modified (e.g., by rolling) so to reduce the internal end region diameter 142′, 142″ relative to the internal end region diameter 142 in FIG. 5A. In these embodiments, the internal end region diameters 142′, 142″ are each less than the internal barrel diameter 144. In some embodiments, where the second contact point 167 of FIGS. 4G and 4H is between the plunger 104 and the rearward end 129″ of FIG. 5C with the internal end region diameter 142″, the external plunger diameter 122 is at least 90% of the internal end region diameter 142″. This facilitates the same tight engagement at the second contact point 167 as was discussed previously with respect to the first contact point 165. In some embodiments, the rearward end 129″ of FIG. 5C includes a rolled edge 140 that has the internal end region diameter 142″. The difference between the rearward end 129′ of FIG. 5B and rearward end 129″ of FIG. 5C is the degree to which the rearward end has been rolled. FIG. 5C is rolled to a greater extent in the rearward end 129″. The inventors of the present disclosure recognized that the rolled edge 140 advantageously results in a smaller internal end region diameter 142″ which can aid in stabilizing the plunger 104 particularly at the rearward end 129 of the barrel region 102. Without the rolled edge 140 of the rearward end 129″, the plunger 104 would only contact the interior surface of the barrel region 102 at the first contact point 165 and thus there would not be as much stability between the plunger 104 and barrel region 102 as the plunger 104 is moved into and out of the outer member.

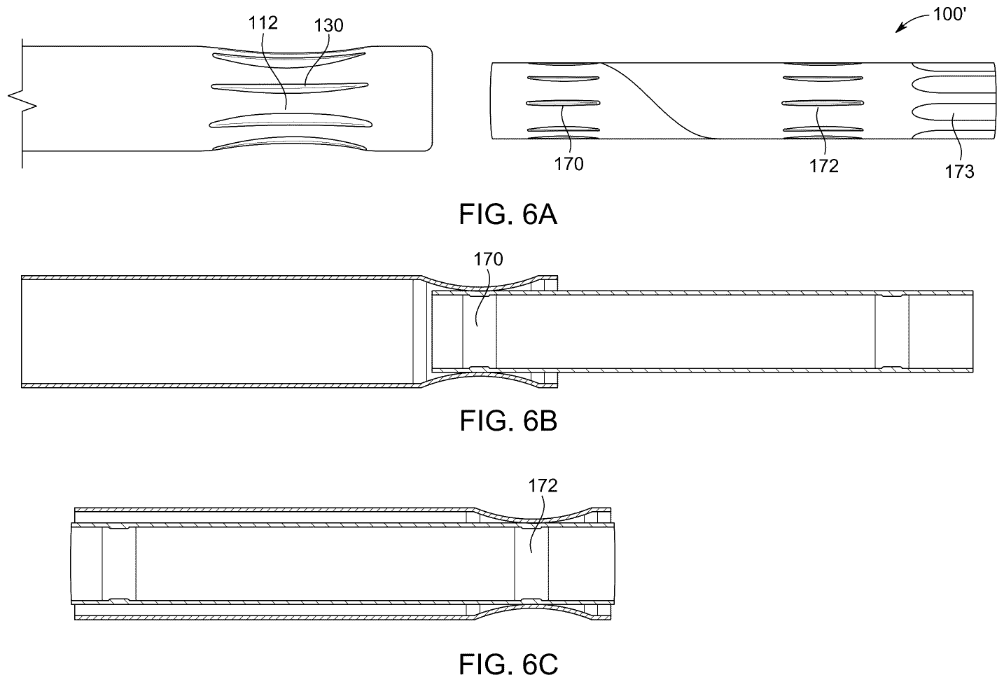

FIGS. 6A through 6C are side views of an applicator 100′ including a barrel region and a plunger with circumferential faces formed in both the barrel region and the plunger. As illustrated in FIG. 6A, the plunger 104 features circumferential faces and/or other markings formed in multiple regions 170, 172, 173. The circumferential faces formed in the multiple regions 170, 172, 173 of the plunger 104 provide physical faces that may cause the plunger 104 to advance differently as the plunger is inserted into the barrel region 102. For example, these multiple regions 170, 172, 173 may result in areas of increased or decreased friction as the plunger advances into the barrel. However, these regions of the plunger 104 are moderate and can be easily overcome by the user applying additional force or less force to the plunger 104. In some embodiments, such as in FIG. 6B, the plunger 104 is inserted within the barrel region 102 until the circumferential faces in the first region 170 engage the grip region 110 of the barrel region 102. FIG. 6C then depicts the plunger 104 after being inserted further within the barrel region 102 until the circumferential faces in the second region 172 engage the grip region 110 and the circumferential faces in the third region 173 are positioned at the end region 129 of the barrel region 102. The circumferential faces (or markings) formed in the multiple regions 170, 172, 173 of the plunger 104 assist the user when inserting the plunger 104 into the barrel region 102 by providing either physical indicators (circumferential faces) or visual indicators (markings) when the plunger 104 has reached various phases of insertion within the barrel region 102. In the embodiments where circumferential faces are formed in the multiple regions 170, 172, 173 of the plunger 104, these circumferential faces are formed in a similar manner (e.g., using the compression device 190 of FIGS. 3A and 3B) as the circumferential faces formed in the barrel region 102.

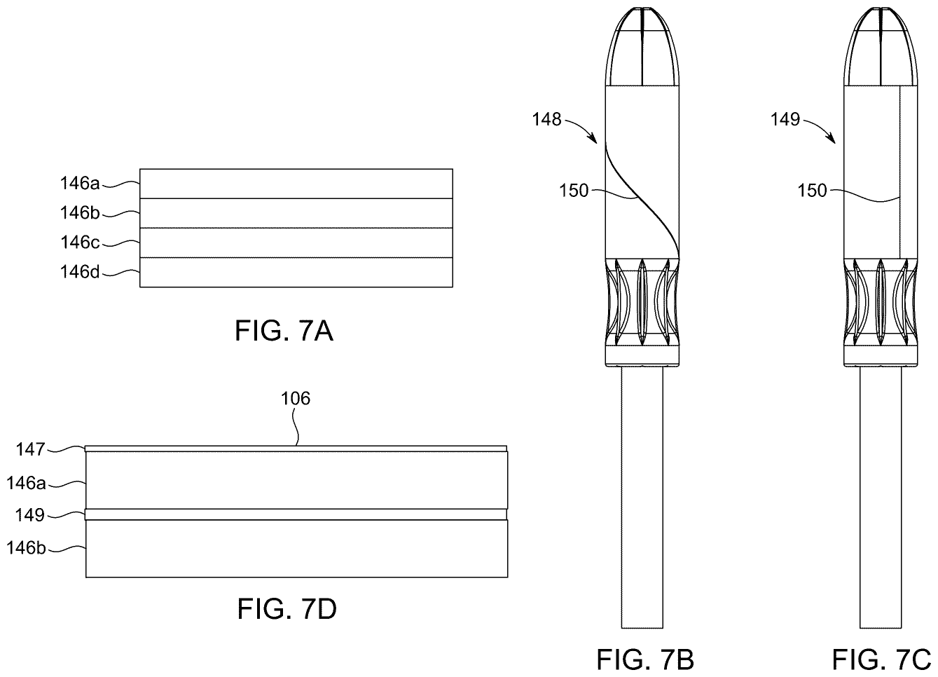

FIG. 7A is a cross-sectional side view of a plurality of material layers 146a through 146d that define the applicator 100. Although four layers 146a through 146d are depicted in FIG. 7A, in other embodiments less or more than four layers may define the applicator 100. The one or more of the layers 146a through 146d may exclude non-naturally occurring fibers. The one or more layers 146a through 146d may include naturally-occurring fibers. Naturally occurring fibers may include cellulosic fibers, wool, silk and other naturally-occurring protein fibers and mixtures thereof, cotton, cotton linters, flax, sisal, abaca, hemp, hesperaloe, jute, bamboo, bagasse, kudzu, corn, sorghum, gourd, agave, loofah, trichomes, seed-hairs, wheat, and mixtures thereof. In other embodiments, each of the one or more layers 146a through 146d include cellulosic material including wood fibers. Wood fiber(s) means fibers derived from both deciduous trees (hereinafter, also referred to as “hardwood”) and coniferous trees (hereinafter, also referred to as “softwood”) may be utilized. Wood fibers may be short (typical of hardwood fibers) or long (typical of softwood fibers). Nonlimiting examples of short fibers include fibers derived from a fiber source selected from the group consisting of Acacia, Eucalyptus, Maple, Oak, Aspen, Birch, Cottonwood, Alder, Ash, Cherry, Elm, Hickory, Poplar, Gum, Walnut, Locust, Sycamore, Beech, Catalpa, Sassafras, Gmelina, Albizia, Anthocephalus, and Magnolia. Nonlimiting examples of long fibers include fibers derived from Pine, Spruce, Fir, Tamarack, Hemlock, Cypress, and Cedar. The one or more of the layers 146a through 146d may be free of synthetic fibers and films.

FIG. 7B is a side view of an applicator including the material layers 146a through 146d of FIG. 7A spirally wound to form the barrel region 102. In some embodiments, the one or more layers 146a through 146d are spirally wound to form at least one of the barrel region 102 and/or plunger 104. FIG. 7B depicts the spiral or non-linear seam 150 formed when the layers 146a through 146d are spirally wound to form the barrel region 102. In some embodiments, one or more of the layers 146a through 146d can include other materials, such as plastic. However, in other embodiments, each of the layers 146a through 146d are 100% cellulosic. In some embodiments, the cellulosic material of the one or more layers 146a through 146d is paper. In other embodiments, the cellulosic material of the one or more layers 146a through 146d is pulp.

FIG. 7C is a side view of an applicator including the material layers 146a through 146d of FIG. 7A convolutely wound to form the barrel region 102. In some embodiments, the one or more layers 146a through 146d are convolutely wound to form at least one of the barrel region 102 and/or plunger 104. FIG. 7C depicts the linear seam 150 formed when the layers 146a through 146d are convolutely wound to form the barrel region 102. In some embodiments, one or more of the layers 146a through 146d can include other materials, such as plastic. However, in other embodiments, each of the layers 146a through 146d are 100% cellulosic. In some embodiments, the cellulosic material of the one or more layers 146a through 146d is paper. In other embodiments, the cellulosic material of the one or more layers 146a through 146d is pulp.

The outer member may be provided in the form of a spirally wound, convolutely wound or other seamed hollow tube which may be formed from paper, paperboard, cardboard or a combination thereof. The paper, paperboard, or cardboard may be manufactured predominantly of wood and/or cellulose pulp fiber. The barrel region 102 may be manufactured to be relatively rigid and have an external barrel diameter 120 of about 10 millimeters to about 20 millimeters. The barrel region 102 has a wall which may be manufactured to have a predetermined thickness of about 0.1 mm to about 0.7 mm. The wall may be constructed from a single layer of material or be formed from two or more plies or layers that are bonded together to form a laminate.

The use of two or more layers enables the manufacturer to use particularly selected materials in the various layers that may enhance the performance of the tampon applicator 100. When two or more layers are included, all the layers may be spirally wound, convolutely wound, and/or longitudinally seamed to form an elongated cylinder. The wall may be constructed using a relatively smooth thin ply of material on the outside or exterior surface that surrounds a coarser and possibly thicker ply. When the wall includes at least three layers, the middle ply may be the thicker ply and the interior and exterior layers may be imparted with a relatively smooth and/or slippery surface finish (e.g., via application of a coating) to facilitate expulsion of the tampon and to facilitate comfortable insertion of the barrel region 102 into the vaginal cavity, respectively. By sandwiching a thick, coarser ply of material between two thin, smooth layers, a barrel region 102 may be provided which is economical and functional. The wall may include one to three, four or five layers, although more layers may be utilized if desired.

The plies, or layers, forming the wall may be held together by a coating, such as an adhesive, or by a coating activated by heat and/or pressure, or by mechanical interaction such as by ultrasonic bonding or pressure bonding, or a combination thereof. The coating may be a petroleum, a petroleum derivative, and/or a plastic with petroleum or petroleum derived components or precursor materials. Thus, an applicator may comprise less than 10 percent by weight or less than 5 percent by weight petroleum, petroleum derivatives and/or plastics with petroleum or petroleum-derived components or precursor materials. For example, each of the layers of the applicator may comprise a cellulosic material, such as paper, and the coating between the layers or plies may be a petroleum, a petroleum derivative, and/or a plastic with petroleum or petroleum derived components or precursor materials. Alternatively, the applicator may comprise a coating, such as an adhesive that is not a petroleum, a petroleum derivative, and/or a plastic with petroleum or petroleum derived components or precursor materials.

The coating may be either water-soluble or water-insoluble. For example, a water-soluble coating, such as a water-soluble adhesive, may be preferred, for reasons of environment-related concerns, so that the layers of the wall will readily separate when wetted by water. Such wetting may occur, for example, upon immersion by flushing in a toilet, or upon exposure to environmental moisture (for example, in an outside landfill in moist or wet conditions). When a water-soluble coating is used, exposure of the barrel region 102 to processes of a municipal waste treatment plant, wherein soaking in water, interaction with chemicals and agitation all occur, will cause the layers of the wall to separate and disperse in a relatively short period of time.

In addition to the objective of providing for dissolution and dispersibility, as noted above, it may be desirable to reduce or eliminate components derived from petroleum according to currently recognized objectives relating to use of sustainably-sourced and environmentally-friendly materials. Many (if not all) adhesives currently used to adhere layers of paper together to form tube structures for applicator components are aqueous formulations including polyvinyl acetate (PVAc). PVAc-based adhesive (exemplified by “white glue” or ELMER'S brand glue) is desirable because it is water soluble, readily penetrates pores of fibrous cellulose/wood pulp paper and adheres to cellulose fibers, is tacky when applied (promoting rapid setting and bonding), and can be formulated with relatively high water content and low viscosity for case of application during manufacturing. However, PVAc is derived from hydrocarbons (acetylene or ethylene), most economically obtained from petroleum. Other non-petroleum-derived adhesive components might be considered, including hide glue and sodium silicate (water glass), used elsewhere in paper-related applications.

When tube stock used to form an applicator or ejection plunger is formed of paper (in turn, formed of cellulose pulp such as wood pulp), the paper may be relatively hydrophilic, porous and absorbent, and also, potentially abrasive to sensitive tissue prior to a coating being applied. The hydrophilic, porous and absorbent qualities may cause the paper material to draw moisture from moist tissues, and thereby increase the potential for irritation and abrasion from sliding contact therebetween. Additionally, these qualities may increase the potential for staining of the paper material with menstrual fluid, which, following withdrawal of an applicator from the vaginal cavity, some users may find undesirable. Accordingly, it may be desirable to apply a coating to the material that, when in melt and/or liquid form readily penetrates the fibrous structure of the paper and seals it to prevent it from absorbing fluid. The coating may be applied to one or more layers or plies, such as the outer layer of the barrel and/or the inner layer of the barrel. The coating may be applied to the layer or ply prior to forming the applicator. More specifically, the coating may be applied prior to the one or more layers being convolutely wound and/or spirally wound.

### L*-a*-b* Color Space

Various embodiments of the present invention will now be discussed that disclose colored paper or cardboard applicators whose outer surface is colored with one or more colors that are not present in conventional cardboard applicators. Although conventional plastic applicators have outer surfaces colored with some of the disclosed colors, these plastic applicators involve an entirely different material (plastic) and process which makes coloring of their outer surfaces fundamentally different than the manufacturing process of paper or cardboard applicators. Thus, the disclosed colored cardboard applicators are not a mere obvious modification of the coloring of conventional plastic applicators. As discussed herein, the inventors of the present invention had to overcome several obstacles in the process of forming the outer surface of the colored cardboard applicators that are unique to the material (e.g., cellulosic) and process for forming cardboard applicators. The inventors recognized that these obstacles are likely the reason why conventional cardboard applicators are not currently available with outer surfaces having the disclosed one or more colors herein.

Prior to discussing the specific embodiments of the colored cardboard applicators, a discussion is provided herein of one or more parameters that are used to characterize color in the human visible spectrum. These parameters are then used to characterize the one or more colors of the disclosed colored cardboard applicators herein.

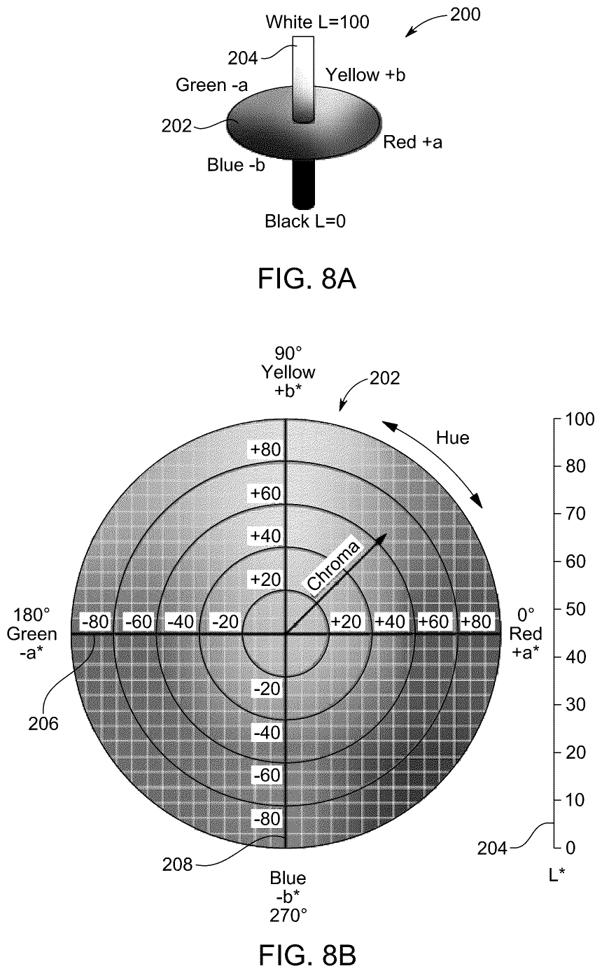

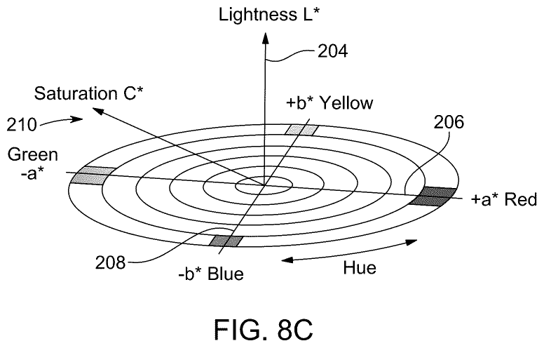

FIG. 8A is a top perspective view of a 3D dimensional space 200 of a L*-a*-b* Color Space (CIELAB color space). As shown in FIG. 8A, the 3D dimensional space 200 includes a 2D dimensional plane 202 and an axis 204 that is orthogonal to the 2D dimensional plane 202. The axis 204 indicates values of a parameter L* that indicates an amount of brightness or shade of a color, where a low value (e.g., 0) represents a darkest shade of the color and a high value (e.g., 100) represents a brightest shade of the color.

FIG. 8B is a top view of 2D dimensional a*-b* plane 202 of the 3D dimensional space 200 of FIG. 8A. As shown in FIG. 8B, the 2D dimensional a*-b* plane 202 includes a first axis 206 that indicates values of a parameter a* which is used to characterize the amount of red and green in a color. Increasingly positive values of a* indicate an increasing amount of red in the color and increasingly negative values of a* indicate an increasing amount of green in the color. As further shown in FIG. 8B, the 2D dimensional a*-b* plane 202 also includes a second axis 208 that indicates values of a parameter b* which is used to characterize the amount of yellow and blue in a color. Increasingly positive values of b* indicate an increasing amount of yellow in the color and increasingly negative values of b* indicate an increasing amount of blue in the color.

FIG. 8C is a side perspective view of the 3D dimensional space 200 of FIG. 8A with axes 206, 208, 204 indicating the a* values, b* values and L* values of the L*-a*-b* Color Space. FIG. 8C depicts a point or vector 210 in the 3D dimensional space 200, where the point 210 is based on the three values of the three parameter coordinates (value of a* along axis 206, value of b* along axis 208 and value of L* along axis 204). In this embodiment, point 210 uniquely represents a specific color.

The L*-a*-b* Color Space (CIELAB color space), also referred to as L*a*b*, is a color space defined by the International Commission on Illumination (CIE) in 1976. It expresses color as three values: L* for perceptual lightness, and a* and b* for the four unique colors of human vision: red, green, blue, and yellow. L* may range from 0 (black) to 100 (white); a* specifies redness-greenness and may range from negative values (green) to positive values (red); and b* specifies yellowness-blueness and may range from negative values (blue) to positive values (yellow). The CIELAB color space is device-independent, meaning that it is not tied to any device or display technology. This makes it a good choice for applications where color needs to be accurately represented on a variety of devices.

The color difference between two colors may be calculated by measuring the L*, a*, b* values for each color. The value of ΔE*, as specified in the following formula, is a measure of the perceived color difference between the two colors. The higher the value of ΔE, the greater the perceived color difference.

$${Δ E = √ ( ( Δ L \text{*)}}^{2} + {( Δ a * )}^{2} + {( Δ b * )}^{2} ) \qquad ( 1 )$$

where ΔL*, Δa*, and Δb* are the differences in the L*, a*, and b* values between the two colors.

$$Δ {L}^{*} = {L}_{1}^{*} - {L}_{2}^{*} \qquad ( 2 )$$

$$Δ {a}^{*} = {a}_{1}^{*} - {a}_{2}^{*} \qquad ( 3 )$$

$$Δ {b}^{*} = {b}_{1}^{*} - {b}_{2}^{*} \qquad ( 4 )$$

### Color Parameters of the Improved Cardboard Applicator

Table 1 below provides average L*, a*, and b* values for conventional plastic applicators having different colors, that were obtained using the Color Measurement Method disclosed herein. Note that unlike the disclosed Color Measurement Method, three sample measurements were taken for each conventional plastic applicator.

TABLE 1

| | | Tampax | Tampax | | Playtex | UbyK | | |
|---|---|---|---|---|---|---|---|---|
| | | Pearl | Pearl Super² | Tampax | Sport | Click | | Playtex |
| | | Super¹ | Adv. Comf. | Radiant | Super⁴ | Super⁵ | This is L | Clean |
| | | Dark | Grip | Super³ | Light | Bright | Super⁶ | Comfort |
| | Sample | Blue | Magenta | Magenta | Green | Pink | White | Clear |
| | No. | Plastic | Plastic | Plastic | Plastic | Plastic | Plastic | Plastic |
| L* | 1 | 22.19 | 31.33 | 31.70 | 63.17 | 43.02 | 64.82 | 72.35 |
| | 2 | 24.97 | 31.92 | 31.41 | 62.80 | 44.13 | 64.63 | 70.78 |
| | 3 | 23.31 | 31.35 | 31.60 | 62.23 | 43.80 | 64.61 | 69.56 |
| L* Average: | | 23.49 | 31.53 | 31.57 | 62.73 | 43.65 | 64.69 | 70.90 |
| a* | 1 | 1.85 | 27.04 | 26.13 | −11.62 | 36.02 | −0.10 | 0.41 |
| | 2 | 0.83 | 27.05 | 26.61 | −11.61 | 35.45 | −0.32 | 0.27 |
| | 3 | 1.83 | 27.09 | 26.72 | −11.75 | 35.71 | −0.20 | 0.57 |
| a* Average: | | 1.50 | 27.06 | 26.49 | −11.66 | 35.73 | −0.21 | 0.42 |
| b* | 1 | −29.03 | −17.86 | −17.65 | 16.70 | −13.24 | 0.55 | 3.84 |
| | 2 | −28.28 | −17.74 | −17.82 | 16.76 | −12.88 | 0.22 | 3.68 |
| | 3 | −29.13 | −17.76 | −17.89 | 17.00 | −13.01 | −0.50 | 3.62 |

| | | | | | | | |
|---|---|---|---|---|---|---|---|
| b* Average: | −28.81 | −17.79 | −17.79 | 16.82 | −13.04 | 0.09 | 3.71 |

¹lot #2202243042W 17:29 1
²lot #1335243032W 18:06 3
³lot #2351243064W 23:52 2
⁴lot # not on wrapper
⁵lot # not on wrapper
⁶lot #2:930243042W 04:09

Table 2 below provides average L*, a*, and b* values for conventional cardboard applicators having different colors, that were obtained using the above discussed Color Measurement Method.

TABLE 2

| | | Tampax | Equate | | |
|---|---|---|---|---|---|
| | | Cardboard | Cardboard | Top (The Organic | Playtex Beyond |
| | Sample | Super⁷ | Super⁸ | Project), Regular⁹ | Cardboard, Super¹⁰ |
| | No. | White | White | White | Light Pink |
| L* | 1 | 80.50 | 82.56 | 82.25 | 72.55 |
| | 2 | 80.83 | 83.72 | 82.17 | 72.25 |
| | 3 | 81.09 | 82.64 | 81.51 | 72.67 |
| L* Average: | | 80.81 | 82.97 | 81.98 | 72.49 |
| a* | 1 | 0.84 | −0.58 | −0.34 | 18.30 |
| | 2 | 0.88 | −0.58 | −0.36 | 18.54 |
| | 3 | 0.81 | −0.59 | −0.34 | 18.18 |
| a* Average: | | 0.84 | −0.58 | −0.35 | 18.34 |
| b* | 1 | 3.52 | 4.93 | 5.36 | 8.78 |
| | 2 | 3.63 | 4.65 | 5.24 | 9.12 |
| | 3 | 3.49 | 5.06 | 5.09 | 8.52 |

| | | | | |
|---|---|---|---|---|
| b* Average: | 3.55 | 4.88 | 5.23 | 8.81 |

⁷lot #2202243039W 11:59
⁸lot #2036.19.06
⁹lot #337 18 1
¹⁰lot # none on box or wrapper

Based on the above Table 2 data that discloses various ranges for the L*, a*, and b* parameters for conventional cardboard applicators, the inventors of the present invention developed various ranges for the L*, a*, and b* parameters for improved cardboard applicators with different colors.

The design of the improved colored cardboard applicator is now discussed herein, with respect to the selection of L* values for the colors used in the improved cardboard applicator. In reviewing the average L* values of conventional cardboard applicators in Table 2 above, the inventors of the present invention recognized that conventional plastic applicators feature colors having an average L* value above 65. Thus, the inventors of the present invention recognized that there is a vacancy in the availability of colored cardboard applicators having a L* value less than 65. Hence, in some embodiments, the colored cardboard applicators disclosed herein are manufactured with colors having a L* value that is less than 65.

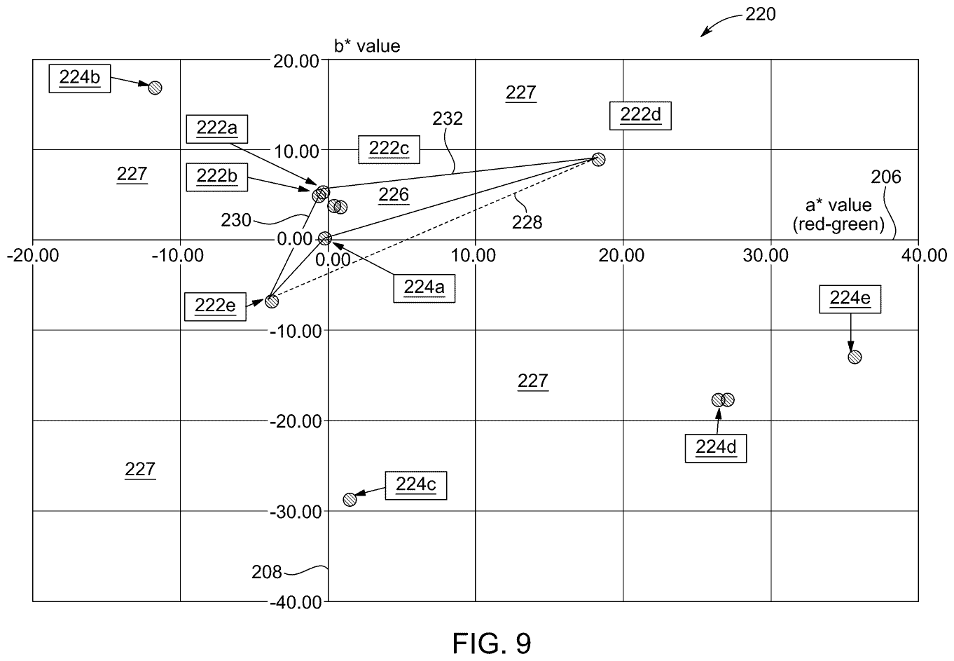

The design of the improved colored cardboard applicator is now discussed herein, with respect to the selection of a* and b* values for the colors used in the improved cardboard applicator. After obtaining the above average L*, a*, and b* values in Tables 1 and 2 for conventional plastic applicators and conventional cardboard applicators, the inventors of the present invention generated a graph to visualize these average a* and b* values in the 2D dimensional a-b plane 202. FIG. 9 is a graph 220 that indicates these average a-b values of the conventional plastic and cardboard applicators (from Tables 1-2). The average a* and b* values for conventional cardboard applicators are indicated by locations 222a through 222e in the graph 220. The average a* and b* values for conventional plastic applicators are indicated by locations 224a through 224e in the graph 200.

The inventors of the present invention, in viewing the graph 220 of FIG. 9, recognized that although the average a* and b* values for conventional plastic applicators (locations 224a through 224e) are distributed over a wide area of the 2D dimensional a-b plane 202, the average a* and b* values for conventional cardboard applicators (locations 222a through 222e) are confined to a relatively small region 226. As shown in FIG. 9, this small region 226 is defined by a first line segment 228 that joins locations 222d, 222e; a second line segment 230 that joins locations 222b, 222e and a third line segment 232 that joins locations 222b, 222d. Thus, the inventors recognized that there is a large region 227 of the 2D dimensional a-b space, defined as outside the small region 226, where there is a large vacancy in the availability of colored cardboard applicators. Hence, in some embodiments, the colored cardboard applicators disclosed herein are manufactured with colors having a* and b* values in this large region 227 and outside the small region 226.

The inventors of the present invention also recognized that the average a* values in Table 2 for conventional colored applicators are between about −4 and 19. Hence, there is a vacancy in the availability of colored cardboard applicators having an a* value above about 19 and/or below about −5. Thus, in some embodiments the colored cardboard applicators disclosed herein are manufactured with colors having an a* value that is greater than about 19 and/or less than about −5.

The inventors of the present invention also recognized that the average b* values in Table 2 for conventional colored applicators are between about −7 and 9. Hence, there is a vacancy in the availability of colored cardboard applicators having a b* value above about 9 and/or below about −7. Thus, in some embodiments the colored cardboard applicators disclosed herein are manufactured with colors having a b* value that is greater than about 9 and less than about −8.

In another embodiment, the inventors of the present invention also recognized that the average a* and b* values in Table 2 for conventional colored applicators are such that when the average a* value is greater than zero, the average b* value is also greater than zero. Hence, the inventors recognized that there is a vacancy in the availability of colored cardboard applicators having a* and b* values such that when the a* value is greater than zero, the b* value is less than zero. Thus, in some embodiments the colored cardboard applicators disclosed herein are manufactured with colors having an a* value greater than zero and a b* value that is less than zero.

As previously discussed, the inventors of the present invention, viewing the graph 220 of FIG. 9, noticed that there is a vacancy in the availability of colored cardboard applicators with a* and b* values outside the small region 226, i.e., within the larger region 227 defined outside the small region 226. Thus, in some embodiments the colored cardboard applicators disclosed herein are manufactured with colors where the a* value and the b* value are outside a boundary of the small region 226 defined by:

$${b}^{*} value = 3.75 \times {a}^{*} value + 9.75 \qquad ( 7 )$$

$${b}^{*} value = 0.19 \times {a}^{*} value + 6.19 \qquad ( 8 )$$

$${b}^{*} value = 0.71 \times {a}^{*} value - 5.45 \qquad ( 9 )$$

where equation 7 represents the line segment 230 in FIG. 9; equation 8 represents the line segment 232 in FIG. 9 and equation 9 represents the line segment 228 in FIG. 9.

In another embodiment, instead of defining the a* and b* values of the improved colored cardboard applicator as those values within the region 227 using equation 7-9, the a* and b* values of the improved colored cardboard applicator are defined such that the b* value satisfies any one of:

$${b}^{*} value > 3.75 \times {a}^{*} value + 9.75 \qquad ( 10 )$$

$${b}^{*} value > 0.19 \times {a}^{*} value + 6.19 \qquad ( 11 )$$

$${b}^{*} value < 0.71 \times {a}^{*} value - 5.45 \qquad ( 12 )$$

Indeed equation 10 indicates those b* values that are greater than the b* values of the line segment 230 in FIG. 9; equation 11 indicates those b* values that are greater than the b* values of the line segment 232 of FIG. 9 and equation 12 indicates those b* values that are less than the b* values of the line segment 228 of FIG. 9.

The above-identified values for the paper or cardboard applicator allow the paper or cardboard applicator to appear more plastic-like. Further, the above-identified values allow for increased functionality of the paper or cardboard applicator by providing application colors that allow for masking of menstrual fluid that may transfer onto the applicator and/or colors that are more readily identifiable to aid with the insertion and removal process. Further, the different in colors between the outer member and the inner member may provide for guidance to the user on depth of insertion and proper alignment of the inner and outer members prior to insertion. By having paper or cardboard applicators include colors as identified in the color spaces previously discussed herein, the paper or cardboard have increased ease of use and functionality of the user.

Additionally, plastic applicators have traditionally had a glossy exterior surface. This glossy exterior surface is perceived by the consumer as being smooth and sleek which leads to the perception that the plastic applicator is more easily inserted and removed during use. The glossy surface also may aid in the masking of menstrual fluid during use. Table 3 includes the gloss data for currently marketed plastic applicators, and Table 4 includes the gloss data for currently and previously marketed cardboard applicators.

In addition to the color features of the improved cardboard applicator (e.g., values of the L*, a*, b* parameter) previously discussed herein, the improved cardboard applicator is provided with generally an increased gloss level to more closely resemble a plastic applicator.

Table 5 below provides an average gloss level for the plastic material used to form conventional plastic applicators. The data was generated using the Gloss Measurement Method disclosed herein.

TABLE 5

| | | | Tampax | | | | | |
|---|---|---|---|---|---|---|---|---|
| | | | Pearl | | | | | |
| | | Tampax | Super² | | Playtex | UbyK | | |
| | | Pearl | Adv. | Tampax | Sport | Click | | Playtex |
| | | Super¹ | Comf. | Radiant | Super⁴ | Super⁵ | This is L | Clean |
| | | Dark | Grip | Super³ | Light | Bright | Super⁶ | Comfort |
| | Sample | Blue | Magenta | Magenta | Green | Pink | White | Clear |
| | No. | Plastic | Plastic | Plastic | Plastic | Plastic | Plastic | Plastic |
| Gloss (60°) | 1 | 80.3 | 46.0 | 53.1 | 15.0 | 5.0 | 76.8 | 9.8 |
| | 2 | 72.8 | 54.9 | 41.9 | 15.5 | 4.7 | 70.6 | 12.2 |
| | 3 | 77.1 | 50.9 | 33.7 | 16.6 | 5.5 | 69.1 | 12.0 |
| | 4 | 68.8 | 50.8 | 48.4 | 15.9 | 4.1 | 65.9 | 10.3 |
| | 5 | 66.1 | 63.7 | 54.6 | 16.4 | 6.2 | 73.9 | 9.8 |
| | 6 | 66.4 | 50.6 | 57.4 | 18.9 | 6.2 | 83.0 | 10.4 |
| | 7 | 70.3 | 51.7 | 58.2 | 15.1 | 6.2 | 71.7 | 10.2 |
| | 8 | 72.7 | 54.8 | 50.4 | 15.9 | 5.6 | 72.1 | 10.0 |
| | 9 | 80.8 | 53.2 | 56.9 | 21.6 | 5.7 | 71.3 | 11.4 |
| | 10 | 76.4 | 52.9 | 55.5 | 20.2 | 6.2 | 76.2 | 11.7 |

| | | | | | | | |
|---|---|---|---|---|---|---|---|
| Gloss Level Average: | 73.2 | 53.0 | 51.0 | 17.1 | 5.5 | 73.1 | 10.8 |

¹lot #2202243042W 17:29 1
²lot #1335243032W 18:06 3
³lot #2351243064W 23:52 2
⁴lot # not on wrapper
⁵lot # not on wrapper
⁶lot #2:930243042W 04:09

TABLE 6

| | | | | Top (The Organic | |
|---|---|---|---|---|---|
| | | Tampax | Equate | Project) | |
| | | Cardboard | Cardboard | Cardboard, | Playtex Beyond |
| | Sample | Super⁷ | Super⁸ | Regular⁹ | Cardboard, Super¹⁰ |
| | No. | White | White | White | Light Pink |
| Gloss | 1 | 29.1 | 33.6 | 25.4 | 34.1 |
| (60°) | 2 | 29.0 | 36.6 | 24.8 | 36.7 |
| | 3 | 23.6 | 28.1 | 25.1 | 29.7 |
| | 4 | 32.0 | 29.7 | 24.3 | 33.9 |
| | 5 | 30.1 | 31.6 | 27.4 | 34.4 |
| | 6 | 26.2 | 34.4 | 26.5 | 39.4 |
| | 7 | 20.7 | 33.0 | 26.9 | 35.9 |
| | 8 | 31.5 | 32.9 | 30.3 | 33.0 |
| | 9 | 28.8 | 29.1 | 30.5 | 32.9 |
| | 10 | 24.9 | 32.1 | 26.9 | 33.9 |

| | | | | |
|---|---|---|---|---|
| Gloss Level Average: | 27.6 | 32.1 | 26.8 | 34.4 |

⁷lot #2202243039W 11:59
⁸lot #2036.19.06
⁹lot #337 18 1
¹⁰lot # none on box or wrapper

The inventors of the present invention recognized that although the gloss measurement levels for conventional plastic applicators varied from about 5 to about 75, the gloss measurement levels for conventional cardboard applicators only varied from about 26 to about 35. Further, most of the plastic applicators had a gloss level average that differed by greater than about 15 from the gloss average of the carboard applicator. Thus, the inventors of the present invention recognized that there is vacancy in the availability of cardboard applicators made from material having a gloss level less than about 26 and/or greater than about 35. Thus, the improved cardboard applicators are made from material having a gloss level that is greater than about 35 and/or less than about 26. In still other embodiments, the improved cardboard applicators are made from material having an inventive color value as specified about in combination with a gloss level that is greater than about 5. In still other embodiments, the improved cardboard applicators are made from material having an inventive color value as specified above in combination with a gloss level that is greater than about 10. In still other embodiments, the improved cardboard applicators are made from material having a gloss level that is greater than about 35. The grip region may have a first gloss level and the barrel region may have a second gloss level, and the first gloss level may be the same as or different than the second gloss level.

In addition to the color features of the improved cardboard applicator (e.g., values of the L*, a*, b* parameter) and the gloss level measurement features previously discussed herein, in other embodiments the improved cardboard applicator is provided with masking features, such as color characteristics to mask the presence of menstrual fluid on one or more portions of the applicator. It may be beneficial for the petal region or insertion region to be colored such that this region provides masking of fluids on the applicator.

Table 7 below lists the above difference in the a* baseline value and the a* value and the Delta E* value for the material of different conventional applicators as determined by the Masking Measurement Method disclosed herein. The color masking method quantifies the ability of a tampon applicator to mask the appearance of any menses or blood that may be left behind on the outer surface of the applicator after the insertion process.

TABLE 7

| | Sample | | | | Average | Delta | Average | Average | Delta |
|---|---|---|---|---|---|---|---|---|---|
| Sample | No. | L* | a* | b* | a* | a* | L* | b* | E* |
| Standard | 1 | 16.99 | 42.27 | 23.47 | 42.1 | na | 16.90 | 23.30 | na |
| Transparent | 2 | 17.03 | 42.60 | 23.85 | | | | | |
| Red Tile + | 3 | 16.80 | 41.83 | 23.08 | | | | | |
| white Pantone | 4 | 16.91 | 42.11 | 23.25 | | | | | |
| chip as | 5 | 16.79 | 41.61 | 22.83 | | | | | |
| backing | | | | | | | | | |
| Playtex | 1 | 16.82 | 42.02 | 23.29 | 42.2 | 0.1 | 16.89 | 23.38 | 0.2 |
| Beyond | 2 | 16.94 | 42.38 | 23.55 | | | | | |
| Cardboard¹⁰ | 3 | 16.86 | 42.02 | 23.43 | | | | | |
| (light pink) | 4 | 16.81 | 42.39 | 23.45 | | | | | |
| | 5 | 17.02 | 42.25 | 23.20 | | | | | |
| Inventive | 1 | 8.92 | 25.43 | 9.65 | 26.0 | −16.1 | 8.90 | 9.87 | 22.4 |
| Sample | 2 | 9.17 | 26.97 | 10.47 | | | | | |
| Cardboard | 3 | 8.81 | 25.68 | 9.63 | | | | | |
| Applicator | 4 | 8.69 | 26.05 | 9.69 | | | | | |
| (blue) | 5 | 8.89 | 25.98 | 9.90 | | | | | |
| Tampax | 1 | 16.28 | 41.48 | 22.64 | 41.1 | −1.0 | 16.12 | 22.32 | 1.6 |
| Cardboard⁷ | 2 | 16.19 | 41.03 | 22.25 | | | | | |
| (white) | 3 | 16.18 | 41.57 | 22.81 | | | | | |
| | 4 | 16.31 | 41.23 | 22.53 | | | | | |
| | 5 | 15.64 | 40.05 | 21.35 | | | | | |
| Tampax Pearl | 1 | 3.57 | 2.40 | 0.74 | 2.5 | −39.6 | 3.60 | 0.70 | 47.5 |
| Plastic¹ | 2 | 3.62 | 2.31 | 0.59 | | | | | |
| (dark blue) | 3 | 3.50 | 2.65 | 0.77 | | | | | |
| | 4 | 3.64 | 2.62 | 0.66 | | | | | |
| | 5 | 3.68 | 2.60 | 0.76 | | | | | |
| Tampax | 1 | 7.79 | 22.97 | 7.88 | 23.5 | −18.6 | 7.95 | 8.10 | 25.6 |
| Radiant | 2 | 7.70 | 23.13 | 7.74 | | | | | |
| Plastic³ | 3 | 8.20 | 24.10 | 8.45 | | | | | |
| (magenta) | 4 | 8.14 | 24.18 | 8.53 | | | | | |
| | 5 | 7.92 | 23.26 | 7.91 | | | | | |

As shown through the data of Table 7, the current market white cardboard applicator by TAMPAX offers little to no masking of the menstrual fluid. Similarly, the light pink cardboard applicator also offers little to no masking of the menstrual fluid. By contrast, the magenta plastic applicator and the dark blue plastic applicator each offer significant masking of menstrual fluid. Similarly, the blue cardboard inventive sample offers significant masking of menstrual fluid as compared to the current market cardboard applicators. Thus, the inventors have identified a need for cardboard applicators to have colors that actively mask menstrual fluid, which is consumer preferable and more closely resembles a plastic applicator. In some embodiments, an inventive carboard applicator may have a delta a* value less than about −1.0 or from about −1.0 to about −20.0 and/or a delta E value of greater than about 4 or from about 4 to about 50 to mask fluid at a greater level than current market cardboard applicator and to resemble the consumer preferred plastic applicators.

In summary, the combination of the color as specified by the *L, *a, *b values disclosed herein, the average gloss level as disclosed herein, and the masking of menstrual fluid have allowed for a cardboard applicator that performs more closely to its plastic counterpart. Further, these variables lead to a cardboard applicator with improved function, such a sleek and smooth finish that allows for easier insertion and removal during use. In some embodiments, the outer member comprises two or more layers and an outer layer of the two or more layers forms at least a portion of the outer surface of the outer member. The outer layer comprises or consists of cellulosic material, and at least a portion of the outer surface of the outer member has a first color having an L* value of less than 65 and a gloss level of greater than 5. Further, in some embodiments, the outer surface may include a second color and the second color may have an L* value of less than 65. Additionally, the delta E* value between the first color and the second color may be greater than about 2. The first color may have an a* value of greater than about 19 or less than about −5.0 and/or the first color may have a b* value of greater than about 9.0 and less than about −8.0. The first color may have an a* value greater than zero and a b* value less than zero. As previously described, the colored cardboard applicator may also have a gloss level or greater than bout 5, or greater than about 10, or greater than about 20, or greater than bout 35. A cardboard applicator with one or more of the aforementioned parameters achieves a more plastic like appearance and performance.

### Structure of the Improved Cardboard Applicator

The structure and method for manufacturing the improved cardboard applicator will now be discussed. The improved cardboard applicator is colored using one or more of the L*, a* and/or b* parameter values that were previously discussed herein.

As shown in FIG. 7D, in some embodiments, the outer member including the barrel region 102 includes two or more layers 146a, 146b. The outer layer 146a forms a portion of the outer surface 106 of the barrel region 102. In some embodiments, the outer layer 146a includes cellulosic material. In some embodiments, the other layers 146b through 146d are made from material other than cellulosic material (e.g., plastic). In still other embodiments, the other layers 146b through 146d are made from cellulosic material and thus the material used to form the barrel region 102 is 100% cellulosic. In still other embodiments, each of the outer member (barrel region 102) and inner member (plunger 104) are made from material that is void of synthetic fibers and films. In some embodiments, the improved cardboard applicators disclosed herein are made from material that is substantially free of material derived from petroleum, such as materials disclosed in U.S. Patent Publication No. 2023/0090472 A1, which is incorporated by reference herein.

In some embodiments, the outer surface 106 of the barrel region 102 has a second portion that includes the second layer 146b of the layers 146a through 146d. In this example embodiment, both the outer layer 146a and the second layer 146b are visible to a consumer on the outer surface 106 of the barrel region 102.

Although FIG. 7A depicts a plurality of layers 146a through 146d defining the material used to form the improved cardboard applicator, in other embodiments the material has a different layer structure. FIG. 7D is a cross-sectional side view of a plurality of material layers 146a and 146b that define the outer member (grip region, insertion region, and barrel region) or inner member (plunger) of the improved cardboard applicator 100. As further shown in FIG. 7D, the material also features a coating 147 on an outer surface of the first layer 146a and an adhesive layer 149 between first and second layers 146a, 146b. The adhesive may be present between any two layers to allow the outer member or the inner member to be configured in a convolutely wound or spirally wound configuration and hold the form of an applicator as described herein. In this embodiment, the outer coating 147 defines the outer surface 106 of the improved cardboard applicator (e.g., barrel region 102 or plunger 104). In some embodiments, the coating 147 comprises wax and/or has a thickness in a range between about 1-3 microns. In some embodiments, the Delta E* value between the color of the adhesive layer 149 and the adjacent layers 146a, 146b is less than about 0.5-0.6 such that the adhesive layer 149 is not perceptible to the human eye. In some embodiments, the coating 147 is a hydrogenated vegetable wax (e.g., high MW oil). In some embodiments, the coating 147 is not colored and instead color is applied to the layer 146a. In some embodiments, the coating 147 advantageously acts as a cover to prevent transfer of the color of the layer 146a to the body or anatomy.

For the above embodiment of FIG. 7D, when the improved cardboard applicator 100 is manufactured, the coating 147 is first applied to the cardboard material defined by the layers 146 after which the cardboard is wound to form the spirally wound layers 148 with the spiral seam 150. Although FIG. 7B depicts the spiral seam 150 in the spirally wound layers 148, in other embodiments the layers of material are convolutely wound.

In some embodiments, one or more features of the layers 146a and 146b of FIG. 7D differs. The outer layer 146a defining the outer surface 106 of the improved cardboard applicator 100 may be a ˜50 gsm coated paper (e.g., with the coating 147). The inventors of the present invention recognized that this layer 146a with the applied coating 147 provides the smooth (low wet-COF) insertion and shiny appearance. In some embodiments, the layer 146a and/or coating 147 are configured based on the one or more parameter values of the gloss level measurement disclosed herein. Additionally, in another example embodiment the inner layers 146b through 146d may be ˜80 gsm paper. Each inner layer 146b through 146d provides wall thickness and structural stability to the material. In some embodiments, the inner layers are uncoated (no coating 147) to improve adhesion with a minimum adhesive layer 149 between adjacent inner layers.

In some embodiments, the first layer 146a of the cardboard material has a first color (e.g., blue, magenta, etc.) and the second layer 146b that is disposed adjacent to the first layer 146a has a second color (e.g., white) that is different from the first color. When winding the layers 146, the spiral wound seams 148 with the seam 150 are formed such that the second color of the second layer 146b is not visible through the seam 150 on the outer surface 106 of the cardboard applicator. In one example embodiment, the Delta E* value across the seam 150 is less than a threshold value for human perception, such as about 1 and/or in a range between about 0.5 to 0.6.

In some embodiments, as shown in FIG. 7D the outer layer 146a defines an exterior outer layer surface 106 that is the outer surface of the improved cardboard applicator 100 and the second layer 146b defines an interior outer layer surface. In this example embodiment, the exterior outer layer surface 106 has the first color and the second interior layer surface has a second color that is different from the first color.

In yet other embodiments, such as shown in FIG. 7A, the material includes a third layer 146c that is disposed adjacent to the second layer 146b. The first layer 146a may have the first color, the second layer 146b may have the second color, and the third layer 146c may have a third color, where the first color, the second color, and the third color are each different colors. In another example embodiment, the second color and the third color are the same color and are each different colors from the first color. For purposes of this description, “different color” means two colors having a minimum Delta E* value (e.g., greater than 2, greater than 4, etc.) that is beyond the minimum Delta E* value for human perception of different colors.

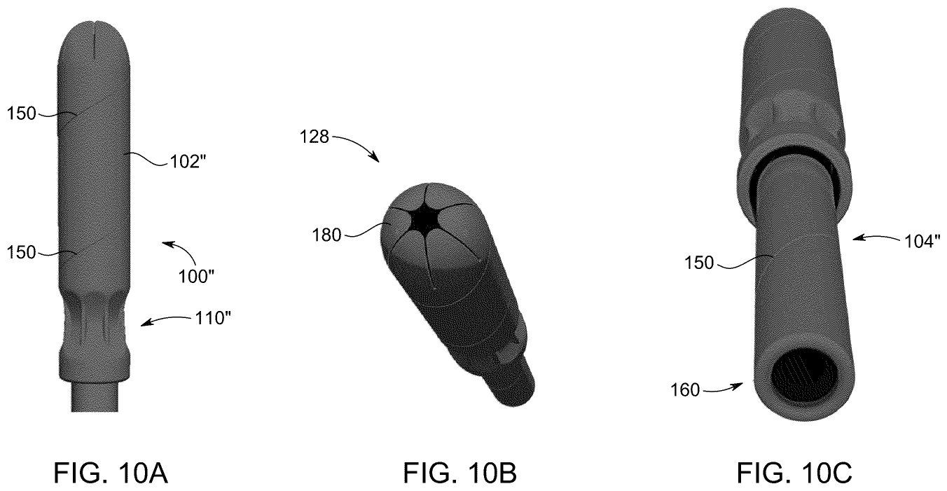

Various views of an improved cardboard applicator are now shown and discussed. FIG. 10A is a side view of a tampon product with a colored applicator 100″ including a barrel region 102″ and a plunger 104″. FIG. 10B is a perspective top view of the forward end 128 of the barrel region 102″ of the colored applicator 100″ of FIG. 10A. FIG. 10C is a perspective bottom view of the rearward end 160 of the plunger 104″ of the colored applicator 100″ of FIG. 10A.

As shown in FIGS. 10A through 10C, in some embodiments the grip region 110″ and/or the forward end 128 of the barrel region 102″ are formed with the first color (e.g., any of the colors based on the previously discussed L*, a*, and/or b* parameter values, such as those disclosed with respect to Tables 3 and 4). In some embodiments, only the grip region 110″ is colored with the first color while the barrel region 102″ is not colored with the first color. In other embodiments, only the barrel region 102″ is colored with the first color and the grip region 110″ is not colored with the first color. In still other embodiments, each of the grip region 110″, the barrel region 102″ and the forward end 128 are each colored with the first color. Thus, the insertion region or the petal region may have different L*, a*, and/or b* parameter values, such as a lower L* value and a higher chroma, than other regions such as the barrel region and/or the grip region.

The manufacturing process for the improved cardboard applicator may include a first step of forming the material including two or more layers 146 (FIG. 7A) which may or may not feature the coating 147 and/or adhesive layers 149 (FIG. 7C). In a first step, the outer layer 146a may be colored based on one or more of the L*, a*, and/or b* parameter values previously disclosed, such as those discussed with respect to Tables 3 and 4. In a second step, the material including the layers 146 are wound to form the outer member (e.g., barrel region 102) and/or inner member (e.g., plunger 104). The inventors recognized that when performing the second winding step, the layers are wound such that there is little to no gap in the spiral seam 150. The inventors recognized that this advantageously ensures that a second color (e.g., white) of the inner layers 146b through 146d is not perceptible to the human eye through the seam 150. In this example embodiment, the second step is performed such that the Delta E* value across the seam 150 is less than about 1 and/or less than about 0.5-0.6. Conventional plastic applicators are not formed using the spiral seam 150, and thus the process for manufacturing plastic applicators cannot be employed or used to modify the process for manufacturing cardboard applicators.

Although FIGS. 7A through 7C and FIGS. 10A through 10C discuss some embodiments of the multiple layer structure of the outer member (e.g., barrel region 102) and coloring of the outer surface of the outer member, in other embodiments the inner member (e.g., plunger 104) can have a similar multiple layer structure as the outer member previously discussed herein. In these embodiments, the inner member (e.g., plunger 104) includes two or more layers 146a, 146b, where an outer layer 146a of the two or more layers 146a, 146b forms at least a portion of an outer surface 106 of the plunger 104. Additionally, as with the previously discussed embodiment regarding the outer member, the outer layer 146a of the inner member includes cellulosic material.

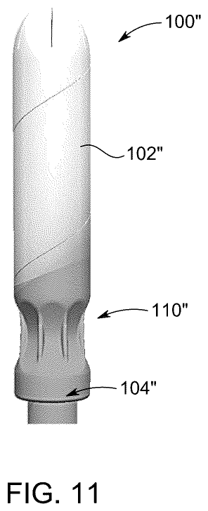

Although the embodiments discussed above with respect to FIGS. 10A through 10C discuss the improved cardboard applicator with the outer surface colored with a single color, in other embodiments, the outer surface of the improved cardboard applicator can be colored with multiple colors. FIG. 11 is a side view of a tampon product with a multi-colored applicator 100′″ including a barrel region 102′″, a plunger 104′″ and a grip region 110′″. In some embodiments, the barrel region 102′″ is colored with a first color and the grip region 110′″ is colored with a second color that is different from the first color. In this example embodiment, the first and second colors have L*, a*, b* parameter values within the ranges as previously disclosed herein. In some embodiments, the first and second colors are different such that the Delta E* value is greater than about 2 and less than about 128. In still another example embodiment, the Delta E* value between the first and second colors is greater than about 4. The applicator may also include a color gradient such that the color doesn't abruptly change between different regions of the applicator, such as the grip region and the barrel region and the insertion region, but rather changes gradually between or withing the various regions of the applicator.

Alternatively, the distinction between the barrel region and the grip region may be abrupt such that the barrel regio and the grip region are two distinctly different colors and/or two distinctly different gloss levels.

Although FIG. 11 depicts different portions of the outer member (e.g., the barrel region 102′″ and the grip region 110′″) having different respective first and second colors, in other embodiments different portions of the inner member (e.g., plunger 104′″) can be colored with different first and second colors. In some embodiments, at least a portion of the outer surface of the inner member has a second color that is different from a first color of the outer member (e.g., barrel region 102′″). In still other embodiments, two different portions of the plunger 104′″ have different first and second colors, where the first and second colors each have an L* value of less than 65. In still other embodiments, three different portions of the plunger 104′″ have different first, second and third colors, where the first, second and third colors each have an L* value of less than 65. This multi-colored inner member (e.g., plunger 104′″) would resemble a color variation as shown for the barrel region 102′″ in FIG. 11 except the multiple colors are provided on the plunger 104″.

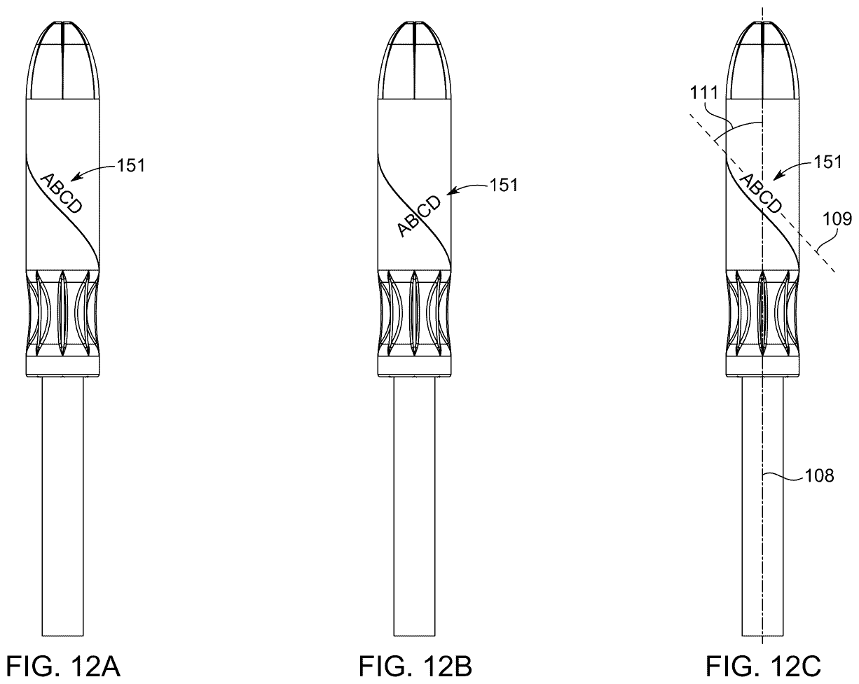

In addition to the outer surface of the improved cardboard applicator having one or more colors, indicia (e.g., print indicia, and/or tactile indicia) can be formed on the outer surface of the improved cardboard applicator. FIG. 12A is a side view of a tampon product with a colored applicator and having indicia 151 on an outer surface 106 thereof. FIG. 12B is a side view of a tampon product with a colored applicator with indicia 151 on an outer surface 106 thereof and extending across the seam 150 defining the outer surface. In some embodiments, the indicia 151 is printed indicia (e.g., that provide information to the consumer during use of the applicator, indicating a trademark or brand name of the product, etc.). As shown in FIG. 12A, in some embodiments the indicia 151 is printed on the outer surface 106 so to be substantially parallel to the seam 150. As shown in FIG. 12B, in another embodiment the indicia 151 is printed on the outer surface 106 so that it extends across a portion of the seam 150. Although FIGS. 12A through 12C depict the spiral shaped seam 150, in other embodiments the seam is axial. Although not depicted in FIGS. 12A through 12C, in some embodiments, the indicia 151 is printed on the outer surface 106 such that it is substantially perpendicular to the longitudinal applicator axis 108. For the embodiment of FIG. 12A where the printed indicia 151 is oriented parallel to the spiral seam 150, FIG. 12C depicts that the printed indicia 151 forms a print angle 111 relative to the longitudinal applicator axis 108 and may or may not be parallel to the spiral seam. In some embodiments, the print angle 111 is greater than about 5 degrees or greater than about 15 degrees or greater than about 30 degrees or from about 0 degrees to about 90 degrees or from about 5 degrees to about 90 degrees or from about 15 degrees to about 45 degrees.

### Test Methods

### Color Measurement Method

After defining the Color Label Space and the parameters (L*, a*, b*) that are used to define each color, a color measurement method is now disclosed herein which is used to measure these parameter values for the materials used to make colored tampon applicators or colored plastic applicators. This method is employed to measure the parameters of conventional plastic applicators and conventional cardboard applicators, as well as the improved colored cardboard applicators disclosed herein.

Color measurements are made on tampon applicators using a 45°/0° spectrophotometer equipped with a 2 mm diameter measurement aperture and capable of making standard CIE L*a*b* measurements in accordance with ASTM E1349. An example of a suitable spectrophotometer is the handheld X-rite eXact® (available from X-Rite®, Inc., Grand Rapids, MI) or equivalent. The analyst wears gloves (e.g., nitrile, or other disposable laboratory gloves) at all times while handling the test sample to prevent the transfer of any contaminant or skin oils to the surface of the test sample. All testing is performed in a room maintained at a temperature of 23° C.±2.0° C. and a relative humidity of 50%±2% and test samples are conditioned under the same environmental conditions for at least 2 hours prior to testing.

For this procedure, the test sample is the tampon applicator with the tampon core left in place inside the barrel portion of the applicator. To prepare a test sample, a tampon applicator is removed from its wrapper but the tampon core is not expelled from the applicator. In the event that the tampon applicator to be tested does not have a tampon core present, the color measurement is executed using the empty tampon applicator as the test sample. In like fashion, a total of five replicate test samples are prepared.

Prior to making any color measurements, the spectrophotometer is calibrated per the vendor instructions and a 2 mm diameter measurement aperture is installed. The spectrophotometer is configured to use the CIE L*a*b* color space with a C standard illumination, a 10° observer, and no UV filter (i.e., MO measurement condition for the X-rite eXact®). Color measurements are performed as follows. The test sample is placed onto a rigid, horizontal benchtop such that the barrel portion of the applicator has its longitudinal axis parallel to the surface of the benchtop. The handheld spectrophotometer is positioned above the test sample such that the measurement aperture is centered over the longitudinal midpoint of the barrel portion of the applicator. Regions near the petals (i.e., insertion end) or grip portion of the applicator as well as any seams or surface defects are excluded from the measurement location. Slight pressure is then applied to the test sample against the aperture of the handheld spectrophotometer to ensure the surface of the test sample is slightly flattened and flush with the aperture. The measurement aperture is properly positioned against the test sample when there are no gaps present for light to escape between the perimeter of the measurement aperture and the test sample, and the surface of the test sample is slightly flattened but not wrinkled or crimped. A reading is taken and the L*, a*, and b* values are recorded to the nearest 0.1 unit. In like fashion, these steps are repeated until the L*, a*, and b* values for all five replicate test samples have been recorded.

The arithmetic mean is then calculated for the L* values recorded for all five replicate test samples and reported as L* to the nearest 0.1 unit. In like fashion, the arithmetic mean for the a* values and then the b* values are calculated and reported as a* and b*, respectively, to the nearest 0.1 unit.

The color difference between two test samples, or between two regions on a given test sample, can be quantified by calculating a Delta E* value. The L*, a*, and b* values measured for a first sample (or region) are compared to the L*, a*, and b* values measured for a second sample (or region) using the following equation, which his similar to equation 1:

$$Delta {E}^{*} ( 1976 ) = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}} \qquad ( 5 )$$

The Delta E* value is reported to the nearest 0.1 units.

A chroma value is then calculated using the measured a* and b* values obtained for a given sample using the following equation:

$$chroma = \sqrt{{a}^{* 2} + {b}^{* 2}} \qquad ( 6 )$$

The chroma value is reported to the nearest 0.1 units.

### Gloss Measurement Method

Gloss measurements of tampon applicators are made using a 60° geometry gloss meter equipped with a 2 mm diameter measurement aperture capable of making measurements on curved surfaces. An example of a suitable 60 degree geometry gloss meter is the benchtop Novo-Curve® (available from Rhopoint Americas®, Inc., Troy, MI), or equivalent. The analyst wears gloves (e.g., nitrile, or other disposable laboratory gloves) at all times while handling the test sample to prevent the transfer skin oils or other potential contaminants to the surface of the test sample. All testing is performed in a room maintained at a temperature of 23° C.±2.0° C. and a relative humidity of 50%±2% and test samples are conditioned under the same environmental conditions for at least 2 hours prior to testing.

For this procedure, the test sample is the tampon applicator with the tampon core left in place inside the barrel portion of the applicator. To prepare a test sample, a tampon applicator is removed from its wrapper but the tampon core is not expelled from the applicator. In the event that the tampon applicator to be tested does not have a tampon core present, the gloss measurement is to be executed using the empty tampon applicator as the test sample. In like fashion, a total of five replicate test samples are prepared.

Prior to making any gloss measurements, the gloss meter is calibrated per the vendor instructions and a 2 mm diameter measurement aperture is installed. Set the gloss meter to take readings at a 60 degree geometry. Gloss measurements are performed as follows. Place the test sample onto the gloss meter with the measurement location centered over the measurement aperture such that the longitudinal axis of the sample is aligned horizontally and parallel to the surface of the measurement aperture. The measurement location is the longitudinal midpoint of the barrel portion of the applicator. Regions near the petals (i.e., insertion end) or grip portion of the applicator as well as any seams or surface defects are excluded from the measurement location. Very light pressure is applied to the test sample to ensure the measurement aperture is fully covered by the test sample. The test sample is slowly rolled back and forth over the measurement aperture while simultaneously taking continuous gloss measurement readings (i.e., “continuous read mode” for the Novo-Curve gloss meter). Rolling the test sample back and forth over the measurement aperture enables the correct measurement position to be found for the curved surface. The largest (or “peak”) gloss value reading obtained during this measurement process is recorded as gloss to the nearest 0.1 gloss unit (GU). In like fashion, these steps are repeated until gloss values for all five replicate test samples have been recorded. The arithmetic mean is calculated across the gloss values recorded for all five replicate test samples and reported as Gloss level to the nearest 0.1 GU.

### Masking Measurement Method

The color masking method quantifies the ability of a tampon applicator to mask the appearance of any menses or blood that may be left behind on the outer surface of the applicator after the insertion process. Color masking measurements are made using a 45°/0° spectrophotometer equipped with a 2 mm diameter measurement aperture and capable of making standard CIE L*a*b* measurements in accordance with ASTM E1349. An example of a suitable spectrophotometer is the handheld X-rite eXact® (available from X-Rite®, Inc., Grand Rapids, MI) or equivalent. The extent of a tampon applicator to mask the color red is quantified by calculating the difference between the a* baseline value (measured for a standard transparent red tile placed over a white PANTONE® plastic standard chip) and the a* value measured for a standard transparent red tile placed over the tampon applicator test sample. Similarly, the color masking effect can also be quantified by calculating the total color difference (Delta E*) between the baseline color values (L*, a*, b*; measured for a standard transparent red tile placed over a white PANTONE plastic standard chip) and the color values (L*, a*, b*) measured for a standard transparent red tile placed over the tampon applicator test sample. The analyst wears gloves (e.g., nitrile, or other disposable laboratory gloves) at all times while handling the test sample to prevent the transfer of any contaminant or skin oils to the measurement surfaces. All testing is performed in a room maintained at a temperature of 23° C.±2.0° C. and a relative humidity of 50%±2% and test samples are conditioned under the same environmental conditions for at least 2 hours prior to testing.

For this procedure, the test sample is the tampon applicator with the tampon core left in place inside the barrel portion of the applicator. The tampon applicator can be composed of plastic, cardboard, bio-based material, or any combination thereof. To prepare a test sample, the tampon applicator is removed from its wrapper but the tampon core is not expelled from the applicator. In the event that the tampon applicator to be tested does not have a tampon core present, the color measurement is to be executed using the empty tampon applicator as the test sample. In like fashion, a total of five replicate test samples are prepared.

In this procedure, the red color of menses or blood is represented by using a standard transparent red tile. A transparent red tile, such as Acrylite® Premium FF with color reference red 3M031 GT/#2423 extruded acrylic sheet with a thickness of 3.175 mm±0.3 mm and a 5% light transmission (available from Rochm America LLC®, Sanford, ME), or equivalent, is used as the standard transparent red tile. Prior to making any measurements, any paper backing that may be present on the standard transparent red tile is removed. A white PANTONE® plastic standard chip is used to back the transparent red tile to obtain the baseline color values (L*, a*, b*) that all test samples will be compared to when calculating the color difference. The specific white PANTONE® plastic chip to be used is called “bright white” (color designation 11-0601 TCX, available from Pantone® LLC/X-Rite®, Inc, Grand Rapids, Michigan, USA), and has color coordinate values of L*−96.34, a*=−1.2 and b*=2.27, or equivalent. The PANTONE® plastic chip has a glossy and matte side, and it is the matte side that is to face the standard transparent red tile when making the color measurements.

Prior to making any color measurements, the spectrophotometer is calibrated per the vendor instructions and a 2 mm diameter measurement aperture is installed. The spectrophotometer is configured to use the CIE L*a*b* color space with a C standard illumination, a 10° observer, and no UV filter (i.e., MO measurement condition for the X-rite eXact®). Color measurements to obtain the baseline color values are performed as follows. The white PANTONE® plastic standard chip is placed onto a rigid, horizontal benchtop such that the matte side of the chip is facing up. The standard transparent red tile is then placed on top of the white plastic chip such that the red tile is centered over the white plastic chip. The handheld spectrophotometer is positioned above the standard red transparent tile such that the measurement aperture is centered over a region that is within the matte portion of the white PANTONE® plastic chip. Regions that lie outside of the matte square on the white PANTONE® plastic chip (i.e., where a hole-punch or printed label may be present) are excluded from the measurement location. A reading is taken to record the baseline color values (L*, a*, and b*) to the nearest 0.1 unit. In like fashion, the measurement is repeated for additional, non-overlapping locations within the matte portion of the white PANTONE® plastic chip until a total of five baseline L*, a*, and b* values have been recorded. The arithmetic mean is calculated for the baseline L* values recorded for all five measurement locations and report as L*1 to the nearest 0.1 unit. In like fashion, the arithmetic mean is calculated for the baseline a* values and then the b* values and reported as a*1 and b*1, respectively, to the nearest 0.1 unit.

Color measurements are made on the tampon applicator test sample as follows. The test sample is placed onto a rigid, horizontal benchtop such that the barrel portion of the applicator has its longitudinal axis parallel to the surface of the benchtop. The standard transparent red tile is then placed on top of the test sample such that it is centered over the test sample. The handheld spectrophotometer is positioned onto the surface of the standard transparent red tile such that the measurement aperture is centered over the longitudinal midpoint of the barrel portion of the applicator. Regions near the petals (i.e., insertion end) or grip portion of the applicator as well as any seams or surface defects are excluded from the measurement location. Slight pressure is applied against the standard transparent red tile and test sample using the measurement aperture of the handheld spectrophotometer to ensure the surface of the test sample is slightly flattened and flush with the surface of the red tile so that when a reading is taken, the entire field of view of the aperture is filled with the test sample covered by the standard transparent red tile. The measurement aperture is properly positioned when there are no gaps present for light to escape between the perimeter of the measurement aperture and the red tile, and the surface of the test sample is slightly flattened but not wrinkled or crimped. A reading is taken to record the L*, a*, and b* values to the nearest 0.1 unit. In like fashion, this step is repeated until the L*, a*, and b* values for all five replicate test samples have been recorded. The arithmetic mean is calculated for the L* values recorded for all five replicate test samples and reported as L*2 to the nearest 0.1 unit.

In like fashion, the arithmetic mean is calculated for the a* values and then the b* values recorded for the test samples and report as a*, and b*2, respectively, to the nearest 0.1 unit.

The difference between the a* baseline value and the a* value of the test sample is quantified by calculating Delta a* using the following equation.

$$Δ {a}^{*} = {a}_{2}^{*} - {a}_{1}^{*} \qquad ( 13 )$$

where a*₁ is the a* baseline value reported for the standard transparent red tile with the white PANTONE® plastic chip as the backing and a*₂ is the a* value reported for the standard transparent red tile with the tampon applicator test sample as the backing. The Delta a* value is reported to the nearest 0.1 units.

The total color difference between the baseline color values and the color values of the test sample is quantified by calculating Delta E* using the following equation.

$$Delta {E}^{*} ( 1976 ) = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}} \qquad ( 14 )$$

where L*₁, a*₁ and b*₁ are the baseline color values reported for the standard transparent red tile with the white PANTONE plastic chip as the backing and L*₂, a*₂ and b*₂ are the color values reported for the standard transparent red tile with the tampon applicator test sample as the backing. The Delta E* value is reported to the nearest 0.1 units.

### Combinations

The following combinations are contemplated herein:

A1. A tampon product comprising: an applicator comprising an outer member and an inner member slidably engaged with the outer member, the outer member comprising an outer surface and a barrel region, wherein the outer member comprises two or more layers, wherein an outer layer of the two or more layers forms at least a portion of the outer surface of the outer member, and wherein the outer layer comprises cellulosic material, and wherein at least a portion of the outer surface of the outer member has a first color, wherein the first color has an L* value of less than 65 and a gloss level of greater than 5.

A2. The tampon product of paragraph A1, wherein the outer member further comprises a grip region.

A3. The tampon product according to any one of the preceding paragraphs, wherein the grip region comprises two or more faces.

A4. The tampon product according to paragraphs A1 or A2, wherein the grip region comprises greater than four faces.

A5. The tampon product of according to paragraph A4, wherein each of the greater than four faces are separated by a rib.

A6. The tampon product according to any one of the preceding paragraphs, wherein the outer member further comprises an insertion end region, and wherein at least one of the grip region and the insertion end region is the first color.

A7. The tampon product according to paragraph A2, wherein at least one of the grip region and the barrel region is the first color.

A8. The tampon product according to paragraph A6, wherein each of the grip region, the barrel region, and the insertion end region is the first color.

A9. The tampon product according to any one of the preceding paragraphs, wherein the outer layer of the two or more layers consists of cellulosic material.

A10. The tampon product according to any one of the preceding paragraphs, wherein the outer layer of the two or more layers consists of cellulosic material and a coating.

A11. The tampon product of according to paragraph A2, wherein the barrel region is the first color and the grip region is a second color, and wherein the first color and the second color are different.

A12. The tampon product according to paragraph A11, wherein a delta E* value of the first color and second color is greater than about 2.

A13. The tampon product according to any one of the preceding paragraphs, comprising a tampon, wherein the tampon comprises a pledget and a cord attached to the pledget, wherein the pledget is disposed within at least a portion of the barrel region of the applicator.

A14. The tampon product according to paragraph A13, wherein the cord is braided.

A15. The tampon product according to any one of the preceding paragraphs, wherein the inner member comprises two or more layers, wherein an outer layer of the two or more layers forms at least a portion of an outer surface of the inner member, and wherein the outer layer comprises cellulosic material.

A16. The tampon product according to paragraph A15, wherein at least a portion of the outer surface of the inner member has a second color, wherein the second color has an L* value of less than 65.

A17. The tampon product according to paragraph A16, wherein at least a portion of the outer surface of the inner member has a third color, and wherein the second color and the third color are different.

A18. The tampon product according to any one of the preceding paragraphs, wherein the outer layer of the outer member comprises printed indicia.

A19. The tampon product according to paragraph A18, wherein the outer member comprises a seam, and wherein the printed indicia is substantially parallel to the seam.

A20. The tampon product according to paragraph A18, wherein the outer member comprises a seam, and wherein the printed indicia extends across a portion of the seam.

A21. The tampon product according to paragraph A18, wherein the printed indicia is disposed on the outer surface at a print angle with respect to a longitudinal applicator axis, wherein the print angle is greater than about 5 degrees.

A22. The tampon product according to any one of the preceding paragraphs, wherein the first color has an a* value of greater than about 19.0 or less than about −5.0.

A23. The tampon according to any one of the preceding paragraphs, wherein the first color has a b* value of greater than about 9.0 or less than about −8.0.

A24. The tampon according to any one of the preceding paragraphs, wherein the first color has an a* value of greater than zero and a b* value less than zero.

B25. A tampon product comprising: an applicator comprising an outer member and an inner member slidably engaged with the outer member, the outer member comprising an outer surface and a barrel region, wherein the outer member comprises two or more layers, wherein an outer layer of the two or more layers forms at least a portion of the outer surface of the outer member, and wherein the outer layer comprises cellulosic material, wherein at least a portion of the outer surface of the outer member has a gloss level of greater than 5, and wherein at least a portion of the outer layer has a first color, wherein the first color has an a* value and a b* value, and wherein the b* value satisfies any one of:

$${b}^{*} value > 3.75 \times {a}^{*} value + 9.75$$

$${b}^{*} value > 0.19 \times {a}^{*} value + 6.19$$

$${b}^{*} value < 0.71 \times {a}^{*} value - 5.45 .$$

B26. The tampon product according to paragraph B25, wherein each of the outer member and the inner member are void of synthetic fibers and films.

C27. A tampon product comprising: an applicator comprising an outer member and an inner member slidably engaged with the outer member, the outer member comprising an outer surface and a barrel region, wherein the outer member comprises two or more layers, wherein an outer layer forms at least a portion of the outer surface of the outer member, and wherein the outer layer comprises cellulosic material, wherein at least a portion of the outer surface of the outer member has a gloss level greater than 5, wherein at least a portion of the outer layer has a first color, wherein the first color has an a* value and a b* value, and wherein the a* value and the b* value are outside a boundary defined by equations:

$${b}^{*} value = 3.75 \times {a}^{*} value + 9.75$$

$${b}^{*} value = 0.19 \times {a}^{*} value + 6.19$$

$${b}^{*} value = 0.71 \times {a}^{*} value - 5.45 .$$

C28. The tampon product according to paragraph C27, wherein the gloss level is greater than 10.

C29. The tampon product according to paragraph C27, wherein the gloss level is greater than 20.

C30. The tampon product according to paragraph C27, wherein the gloss level is greater than 35.

C31. The tampon product according to any one of the preceding paragraphs, wherein the two or more layers comprise a second layer, wherein the second layer is disposed adjacent to the outer layer, wherein the second layer has a second color, and wherein the second color and the first color are different.

C32. The tampon product according to paragraph C31, wherein the outer surface comprises a seam, and wherein the second color of the second layer is not visible at the seam.

C33. The tampon product according to paragraph C31, wherein the outer surface of the outer member comprises a second portion comprising the second layer of the two or more layers.

C34. The tampon product according to paragraph C31, wherein the outer layer has an exterior outer layer surface and an interior outer layer surface, wherein the exterior outer layer surface is the first color and the second interior layer surface is a second color.

C35. The tampon product according to paragraph C34, wherein the first color and the second color are different.

C36. The tampon product according to paragraph C31, wherein the two or more layers comprise a third layer, wherein the third layer is disposed adjacent to the second layer, wherein the third layer has a third color.

C37. The tampon product according to paragraph C36, wherein the second color and the third color are the same.

C38. The tampon product according to any one of the preceding paragraphs, wherein the applicator comprises a grip region adjacent the barrel region, wherein the grip region comprises at least two faces.

C39. The tampon product according to paragraph C38, wherein each of the at least two faces extends from an upper grip region to a lower grip region.

C40. The tampon product according to paragraph C39, wherein each of the at least two faces have a radius of curvature that is about 300 mm.

C41. The tampon product according to paragraph C39, wherein each of the at least two faces have a radius of curvature that is from about 4.3 mm to about 900 mm.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A tampon product comprising:
an applicator comprising an outer member and an inner member slidably engaged with the outer member, the outer member comprising an outer surface and a barrel region,
wherein the outer member comprises two or more layers, wherein an outer layer of the two or more layers forms at least a portion of the outer surface of the outer member, and wherein the outer layer comprises cellulosic material, and
wherein at least a portion of the outer surface of the outer member has a first color, wherein the first color has an L* value of less than 65 and a gloss level of greater than 5.

2. The tampon product of claim 1, wherein the outer member further comprises a grip region.

3. The tampon product of claim 2, wherein the grip region comprises greater than four faces and wherein each of the greater than four faces are separated by a rib.

4. The tampon product of claim 2, wherein the outer member further comprises an insertion end region, and wherein at least one of the grip region and the insertion end region is the first color.

5. The tampon product of claim 2, wherein at least one of the grip region and the barrel region is the first color.

6. The tampon product of claim 4, wherein each of the grip region, the barrel region, and the insertion end region is the first color.

7. The tampon product of claim 1, wherein the outer layer of the two or more layers consists of cellulosic material.

8. The tampon product of claim 1, wherein the outer layer of the two or more layers consists of cellulosic material and a coating.

9. The tampon product of claim 2, wherein the barrel region is the first color and the grip region is a second color, and wherein a delta E* value of the first color and second color is greater than about 2.

10. The tampon product of claim 1, comprising a tampon, wherein the tampon comprises a pledget and a cord attached to the pledget, wherein the pledget is disposed within at least a portion of the barrel region of the applicator.

11. The tampon product of claim 1, wherein the inner member comprises two or more layers, wherein an outer layer of the two or more layers forms at least a portion of an outer surface of the inner member, and wherein the outer layer comprises cellulosic material.

12. The tampon product of claim 11, wherein at least a portion of the outer surface of the inner member has a second color, wherein the second color has an L* value of less than 65.

13. The tampon product of claim 12, wherein at least a portion of the outer surface of the inner member has a third color, and wherein the second color and the third color are different.

14. The tampon product of claim 1, wherein the outer layer of the outer member comprises printed indicia.

15. The tampon product of claim 14, wherein the outer member comprises a seam, and wherein the printed indicia is substantially parallel to the seam.

16. The tampon product of claim 14, wherein the outer member comprises a seam, and wherein the printed indicia extends across a portion of the seam.

17. The tampon product of claim 14, wherein the printed indicia is disposed on the outer surface at a print angle with respect to a longitudinal applicator axis, wherein the print angle is greater than about 5 degrees.

18. The tampon product of claim 1, wherein the first color has an a* value of greater than about 19.0 or less than about −5.0, and wherein the first color has a b* value of greater than about 9.0 or less than about −8.0.

19. The tampon product of claim 1, wherein the first color has an a* value of greater than zero and a b* value less than zero.

20. A tampon product comprising:
an applicator comprising an outer member and an inner member slidably engaged with the outer member, the outer member comprising an outer surface and a barrel region,
wherein the outer member comprises two or more layers, wherein an outer layer of the two or more layers forms at least a portion of the outer surface of the outer member, and wherein the outer layer comprises cellulosic material,
wherein at least a portion of the outer surface of the outer member has a gloss level of greater than 5, and
wherein at least a portion of the outer layer has a first color, wherein the first color has an a* value and a b* value, and wherein the b* value satisfies any one of:
$${b}^{*} value > 3.75 \times {a}^{*} value + 9.75$$
$${b}^{*} value > 0.19 \times {a}^{*} value + 6.19$$
$${b}^{*} value < 0.71 \times {a}^{*} value - 5.45 .$$

21. A tampon product comprising:
an applicator comprising an outer member and an inner member slidably engaged with the outer member, the outer member comprising an outer surface and a barrel region,
wherein the outer member comprises two or more layers, wherein an outer layer forms at least a portion of the outer surface of the outer member, and wherein the outer layer comprises cellulosic material,
wherein at least a portion of the outer surface of the outer member has a gloss level greater than 5,
wherein at least a portion of the outer layer has a first color, wherein the first color has an a* value and a b* value, and
wherein the a* value and the b* value are outside a boundary defined by equations:
$${b}^{*} value = 3.75 \times {a}^{*} value + 9.75$$
$${b}^{*} value = 0.19 \times {a}^{*} value + 6.19$$
$${b}^{*} value = 0.71 \times {a}^{*} value - 5.45 .$$
